(19) United States
(12) Patent Application Publication
(10) Pub. No.: US 2003/0212119 A1
(43) Pub. Date: Nov. 13, 2003

(54) Novel glucagon receptor antagonists/inverse agonists

(76) Inventors: Jesper Lau, Farum (DK); Inge Thoger Christensen, Lyngby (DK); Peter Madsen, Bagsvaerd (DK); Carsten Behrens, Kobenhavn N. (DK)

Correspondence Address: Novo Nordisk Pharmaceuticals, Inc., 100 College Road West, Princeton, NJ, 08540, US

(21) Appl. No.: 10/322,329

(22) Filed: Dec. 18, 2002

Related U.S. Application Data
(60) Provisional application No. 60/342,355, filed on Dec. 20, 2001.

(30) Foreign Application Priority Data
Dec. 20, 2001 (DK) PA 2001 01925

(51) Int. Cl. A61K 31/4184; A61K 31/405; C 07D 4 3/02; A61K 31/415; C07D 209/14

(52) U.S. Cl. 514/381; 514/394; 548/252; 548/304.4; 548/503; 514/419

(57) ABSTRACT

Novel compounds that act to antagonize the action of the glucagon peptide hormone on the glucagon receptor. More particularly, it relates to glucagon antagonists or inverse agonists.

### CROSS REFERENCE TO RELATED APPLICATIONS

The present application claims the benefit under 35 U.S.C. 119 of Danish application PA 2001 01925, filed Dec. 20, 2001, and of U.S. provisional application 60/342,355, filed Dec. 20, 2001, the contents of both of which are hereby incorporated by reference.

### FIELD OF THE INVENTION

The present invention relates to agents that act to antagonize the action of the glucagon peptide hormone on the glucagon receptor. More particularly, it relates to glucagon antagonists or inverse agonists.

### BACKGROUND OF THE INVENTION

Glucagon is a key hormonal agent that, in co-operation with insulin, mediates homeostatic regulation of the amount of glucose in the blood. Glucagon primarily acts by stimulating certain cells (mostly liver cells) to release glucose when blood glucose levels fall. The action of glucagon is opposite to that of insulin, which stimulates cells to take up and store glucose whenever blood glucose levels rise. Both glucagon and insulin are peptide hormones.

Glucagon is produced in the alpha islet cells of the pancreas and insulin in the beta islet cells. Diabetes mellitus is a common disorder of glucose metabolism. The disease is characterized by hyperglycemia and may be classified as type 1 diabetes, the insulin-dependent form, or type 2 diabetes, which is non-insulin-dependent in character. Subjects with type 1 diabetes are hyperglycemic and hypoinsulinemic, and the conventional treatment for this form of the disease is to provide insulin. However, in some patients with type 1 or type 2 diabetes, absolute or relative elevated glucagon levels have been shown to contribute to the hyperglycemic state. Both in healthy control animals as well as in animal models of type 1 and type 2 diabetes, removal of circulating glucagon with selective and specific antibodies has resulted in reduction of the glycemic level. These studies suggest that glucagon suppression or an action that antagonizes glucagon could be a useful adjunct to conventional treatment of hyperglycemia in diabetic patients. The action of glucagon can be suppressed by providing an antagonist or an inverse agonist, ie substances that inhibit or prevent glucagon-induced responses. The antagonist can be peptidic or non-peptidic in nature.

Native glucagon is a 29 amino acid peptide having the sequence: His-Ser-Glu-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-OH

Glucagon exerts its actionby binding to and activating its receptor, which is part of the Glucagon-Secretin branch of the 7-transmembrane G-protein coupled receptor family. The receptor functions by activating the adenylyl cyclase second messenger system and the result is an increase in cAMP levels.

Several publications disclose peptides that are stated to act as glucagon antagonists. Probably, the most thoroughly characterized antagonist is DesHis¹[Glu⁹]-glucagon amide (Unson et al., Peptides 10, 1171 (1989); Post et al., Proc. Natl. Acad. Sci. USA 90, 1662 (1993)). Other antagonists are DesHis¹ ,Phe⁶[Glu⁹]-glucagon amide (Azizh et al., Bioorganic & Medicinal Chem. Lett. 16, 1849 (1995)) and NLeu⁹,Ala^{11,16}-glucagon amide (Unson et al., J. Biol. Chem. 269 (17), 12548 (1994)).

Peptide antagonists of peptide hormones are often quite potent. However, they are generally known not to be orally available because of degradation by physiological enzymes, and poor distribution in vivo. Therefore, orally available non-peptide antagonists of peptide hormones are generally preferred. Among the non-peptide glucagon antagonists, a quinoxaline derivative, (2-styryl-3-[3-(dimethylamino)propylmethylamino]-6,7-dichloroquinoxaline was found to displace glucagon from the rat liver receptor (Collins, J. L. et al., Bioorganic and Medicinal Chemistry Letters 2(9):915-918 (1992)). WO 94/14426 (The Wellcome Foundation Limited) discloses use of skyrin, a natural product comprising a pair of linked 9,10-anthra-cenedione groups, and its synthetic analogues, as glucagon antagonists. U.S. Pat. No. 4,359,474 (Sandoz) discloses the glucagon inhibiting properties of 1-phenyl pyrazole derivatives. U.S. Pat. No. 4,374,130 (Sandoz) discloses substituted disilacyclohexanes as glucagon inhibiting agents. WO 98/04528 (Bayer Corporation) discloses substituted pyridines and biphenyls as glucagon antagonists. U.S. Pat. No. 5,776,954 (Merck & Co., Inc.) discloses substituted pyridyl pyrroles as glucagon antagonists and WO 98/21957, WO 98/22108, WO 98/22109 and U.S. Pat. No. 5,880,139 (Merck & Co., Inc.) disclose 2,4-diaryl-5-pyridylimidazoles as glucagon antagonists. Furthermore, WO 97/16442 and U.S. Pat. No. 5,837,719 (Merck & Co., Inc.) disclose 2,5-substituted aryl pyrroles as glucagon antagonists. WO 98/24780, WO 98/24782, WO 99/24404 and WO 99/32448 (Amgen Inc.) disclose substituted pyrimidinone and pyridone compounds and substituted pyrimidine compounds, respectively, which are stated to possess glucagon antagonistic activity. Madsen et al. (J. Med. Chem. 41, 5151-7 (1998)) discloses a series of 2-(benzimidazol-2-ylthio)-1-(3,4-dihydroxyphenyl)-1-ethanones as competitive human glucagon receptor antagonists. WO 99/01423 and WO 00/39088 (Novo Nordisk A/S) disclose different series of alkylidene hydrazides as glucagon antagonists/inverse agonists. WO 00/69810 (Novo Nordisk A/S) discloses a further class of glucagon antagonists.

These known glucagon antagonists differ structurally from the present compounds.

### Definitions

The following is a detailed definition of the terms used to describe the compounds of the invention:

“Halogen” designates an atom selected from the group consisting of F, Cl, Br and I.

The term “C₁₋₆-alkyl” as used herein represents a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₂₋₆-alkenyl” as used herein represents a branched or straight hydrocarbon group having from 2 to 6 carbon atoms and at least one double bond. Examples of such groups include, but are not limited to, vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, 5-hexenyl and the like.

The term “C₂₋₆-alkynyl” as used herein represents a branched or straight hydrocarbon group having from 2 to 6 carbon atoms and at least one triple bond. Examples of such groups include, but are not limited to, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 2,4-hexadiynyl and the like.

The term “C₁₋₆-alkoxy” as used herein refers to the radical —O—C₁₋₆-alkyl, wherein C₁₋₆-alkyl is as defined above. Representative examples are methoxy, ethoxy, n-propoxy, iso-propoxy, butoxy, sec-butoxy, tert-butoxy, pentoxy, isopentoxy, hexoxy, isohexoxy and the like.

The term “C₁₋₆-alkylthio” as used herein refers to the radical —S—C₁₋₆-alkyl, wherein C₁₋₆-alkyl is as defined above. Representative examples include, but are not limited to, methylthio, ethylthio, n-propylthio, isopropylthio, butylthio, isobutylthio, sec-butylthio, tert-butylthio, n-pentylthio, isopentylthio, neopentylthio, tert-pentylthio, n-hexylthio, isohexylthio and the like.

The term “C₁₋₆-alkanoyl” as used herein refers to the radical —C(O)H or —C(O)C₁₋₅-alkyl, wherein C₁₋₅-alkyl is a saturated, branched or straight hydrocarbon group having from 1 to 5 carbon atoms. Representative examples include, but are not limited to, formyl, acetyl, propionyl, butanoyl, pentanoyl, hexanoyl and the like.

The term “C₃₋₈-cycloalkyl” as used herein represents a saturated, carbocyclic group having from 3 to 8 carbon atoms. Representative examples are cyclopropyl, cyclobutyl, cyclopentyl, cyclohexyl, cycloheptyl, cyclooctyl and the like.

The term “C₄₋₈-cycloalkenyl” as used herein represents a non-aromatic, carbocyclic group having from 4 to 8 carbon atoms containing one or two double bonds. Representative examples are 1-cyclopentenyl, 2-cyclopentenyl, 3-cyclopentenyl, 1-cyclohexenyl, 2-cyclohexenyl, 3-cyclohexenyl, 2-cycloheptenyl, 3-cycloheptenyl, 2-cyclooctenyl, 1,4-cyclooctadienyl and the like.

The term “heterocyclyl” as used herein represents a non-aromatic 3 to 10 membered ring containing one or more heteroatoms selected from nitrogen, oxygen and sulfur and optionally containing one or two double bonds. Representative examples are pyrrolidinyl, piperidyl, piperazinyl, morpholinyl, thiomorpholinyl, aziridinyl, tetrahydrofuranyl and the like.

The term “aryl” as used herein is intended to include carbocyclic, aromatic ring systems such as 6 membered monocyclic and 9 to 14 membered bi- and tricyclic, carbocyclic, aromatic ring systems. Representative examples are phenyl, biphenylyl, naphthyl, anthracenyl, phenanthrenyl, fluorenyl, indenyl, azulenyl and the like. Aryl is also intended to include the partially hydrogenated derivatives of the ring systems enumerated above. Non-limiting examples of such partially hydrogenated derivatives are 1,2,3,4-tetrahydronaphthyl, 1,4-dihydronaphthyl and the like.

The term “aryloxy” as used herein denotes a group —O-aryl, wherein aryl is as defined above.

The term “arylthio” as used herein denotes a group —S-aryl, wherein aryl is as defined above.

The term “aroyl” as used herein denotes a group —C(O)-aryl, wherein aryl is as defined above.

The term “heteroaryl” as used herein is intended to include aromatic, heterocyclic ring systems containing one or more heteroatoms selected from nitrogen, oxygen and sulfur such as 5 to 7 membered monocyclic and 8 to 14 membered bi- and tricyclic aromatic, heterocyclic ring systems containing one or more heteroatoms selected from nitrogen, oxygen and sulfur. Representative examples are furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the ring systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like. “Aryl-C₁₋₆-alkyl”, “heteroaryl-C₁₋₆-alkyl”, “aryl-C₂₋₆-alkenyl” etc. mean C₁₋₆-alkyl or C₂₋₆-alkenyl as defined above, substituted by an aryl or heteroaryl as defined above, for example:

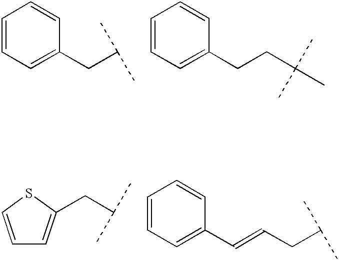

The term “optionally substituted” as used herein means that the groups in question are either unsubstituted or substituted with one or more of the substituents specified. When the groups in question are substituted with more than one substituent the substituents may be the same or different.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

### DESCRIPTION OF THE INVENTION

The present invention relates to a compound of the general formula (I):

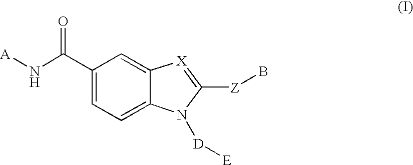

wherein

A is

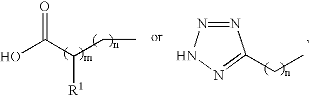

m is 0 or 1,

n is 0, 1, 2 or 3,

with the proviso that m and n must not both be 0,

R¹ is hydrogen, fluoro or —(CH₂)ₒ—OR²,

o is 0 or 1,

R² is hydrogen, C₁₋₆-alkyl, C₁₋₆-alkanoyl, aryl or aryl-C₁₋₆-alkyl,

X is —N═ or —CH═,

B is

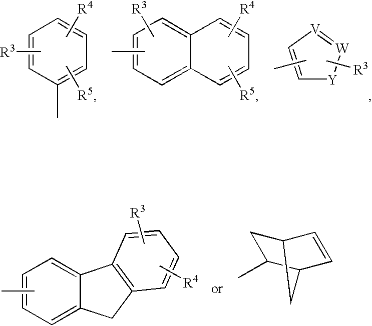

V and W independently are —CH═ or —N═,

Y is —O—, —S— or —NH—,

R³, R⁴ and R⁵ independently are

hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂R⁷, —S(O)_{NR}⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,

C₁₋₆-alkyl, C₂₋₆-alkenyl or C₂₋₆-alkynyl,

which may optionally be substituted with one or more substituents selected from fluoro, —CN, —CF₃, —OCF₃, —OR⁶ and —NR⁶R⁷,

C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,

of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,

aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,

of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,

R⁶ and R⁷ independently are hydrogen or C₁₋₆-alkyl,

or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or two of the groups R³ to R⁵ when placed in adjacent positions together may form a bridge —(CR⁸R⁹)ₛ—O—(CR¹⁰R¹¹)ₜ—O—,

s is 0, 1 or 2,

t is 1 or 2,

R⁸, R⁹, R¹⁰ and R¹¹ independently are hydrogen, C₁₋₆-alkyl or fluoro,

D is —(CH₂)ₚ—,

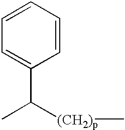

or —(CH₂)ₚ—O—,

p is 0, 1, 2, 3 or 4,

E is

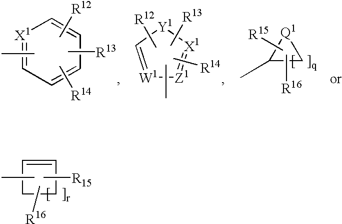

X¹, Z¹ and W¹ independently are —CH═ or —N═,

Y¹ is —O—, —S— or —NH—,

Q¹ is —CH₂— or —NH—,

q is 2, 3, 4, 5 or 6,

r is 1, 2, 3, 4 or 5,

R¹², R¹³ and R¹⁴ independently are

hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —C(O)R¹⁷ or —C(O)OR¹⁷,

C₁₋₆-alkyl, C₂₋₆-alkenyl or C₂₋₆-alkynyl,

which may optionally be substituted with one or more substituents selected from fluoro, —CN, —CF₃, —OCF₃, —OR¹⁷ and —NR¹⁷R¹⁸,

C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,

of which the cyclic moieties may optionally be substituted with one or more substituents selected from fluoro, —C(O)OR¹⁷, —CN, —CF₃, —OCF₃, —OR¹⁷ and —NR¹⁷R¹⁸,

aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,

of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,

R¹⁷ and R¹⁸ independently are hydrogen or C₁₋₆-alkyl,

or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or two of the groups R¹² to R¹⁴ when placed in adjacent positions together may form a bridge —(CR¹⁹R²⁰)ₓ—O—(CR²¹R²²)_{y}—O—,

x is 0, 1 or 2,

y is 1 or2,

R¹⁹, R²⁰, R²¹ and R²² independently are hydrogen, C₁₋₆-alkyl or fluoro,

R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,

wherein the cyclic moieties may optionally be substituted with one or more substituents selected from halogen, —CN, —CF₃, —NO₂, —OR²³, —NR²³R²⁴ and C₁₋₆-alkyl,

R²³ and R²⁴ independently are hydrogen or C₁₋₆-alkyl, or

R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or E is

C₁₋₆-alkyl, C₂₋₆-alkenyl or C₂₋₆-alkynyl,

which may optionally be substituted with one or more substituents selected from halogen, —CN, —CF₃, —OCF₃, —NO₂, —OR²⁵,—SR²⁵, —NR²⁵R²⁶ and C₁₋₆-alkyl,

R²⁵ and R²⁶ independently are hydrogen or C₁₋₆-alkyl, or

R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

Z is —(CR²⁷R²⁸)ᵥ—(O)_{w}—(CR²⁹R³⁰)_{z}—,

v and z independently are 0, 1 or 2,

w is 0 or 1,

R²⁷, R²⁸, R²⁹ and R³⁰ independently are hydrogen or C₁₋₆-alkyl,

with the proviso that the compound must not be

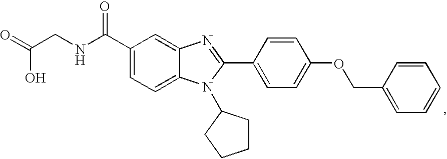

as well as any diastereomer or enantiomer or tautomeric form thereof including mixtures of these or a pharmaceutically acceptable salt thereof.

In one embodiment A is

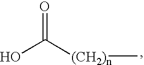

wherein n is as defined for formula (I), such as

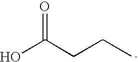

In another embodiment A is

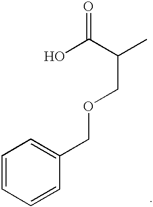

In still another embodiment B is

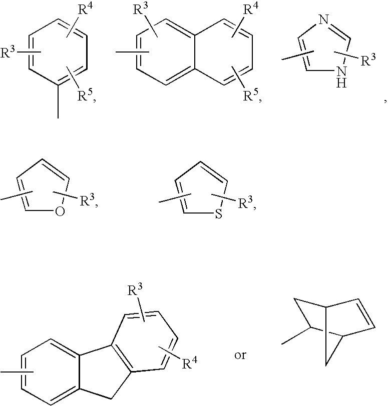

wherein R³ to R⁵ are as defined for formula (I).

In yet another embodiment B is

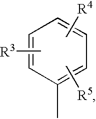

wherein R³, R⁴ and R⁵ independently are

hydrogen, halogen, —CF₃, —OCF₃, —NO₂, —OR⁶, —NR⁶R⁷, C₁₋₆-alkyl,

aryloxy, aryl-C₁₋₆-alkoxy,

of which the cyclic moieties may optionally be substituted with one or more substituents selected from halogen, —CF₃, —OCF₃, —NO₂, —OR⁶, —NR⁶R⁷ and C₁₋₆-alkyl,

R⁶ and R⁷ independently are hydrogen or C₁₋₆-alkyl,

or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

In a further embodiment B is

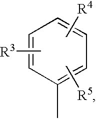

wherein

R³, R⁴ and R⁵ independently are

hydrogen, halogen, —CF₃, —OCF₃, —NO₂, —OR⁶, —NR⁶R⁷, C₁₋₆-alkyl,

phenoxy, phenyl-C₁₋₆-alkoxy,

of which the cyclic moieties optionally may be substituted with one or more substituents selected from halogen, —CF₃, —OCF₃, —NO₂, —OR⁶, —NR⁶R⁷ and C₁₋₆-alkyl,

R⁶ and R⁷ independently are hydrogen or C₁₋₆-alkyl,

or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

In yet a further embodiment B is

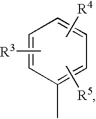

wherein

R³, R⁴ and R⁵ independently are

hydrogen, halogen, —CF₃, —OCF₃, —NO₂, —OR⁶, —NR⁶R⁷, C₁₋₆-alkyl,

phenoxy, phenyl-C₁₋₆-alkoxy,

of which the cyclic moieties may optionally be substituted with one or more substituents selected from halogen, —CF₃, and C₁₋₆-alkoxy,

R⁶ and R⁷ independently are hydrogen or C₁₋₆-alkyl.

In one embodiment R³ is hydrogen, and R⁴ and R⁵ are different from hydrogen.

In another embodiment R³ and R⁴ are hydrogen, and R⁵ is different from hydrogen.

In still a further embodiment B is

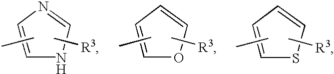

wherein R³ is

hydrogen, halogen, C₁₋₆-alkyl,

aryl, which may optionally be substituted with one or more substituents selected from halogen, —CF₃, —OCF₃, —NO₂, —OR⁶, —NR⁶R⁷ and C₁₋₆-alkyl,

R⁶ and R⁷ independently are hydrogen or C₁₋₆-alkyl,

or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

In yet a further embodiment B is

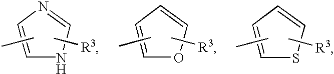

wherein R³ is

hydrogen, halogen, C₁₋₆-alkyl,

phenyl, which may optionally be substituted with one or more substituents selected from halogen, —CF₃, —OCF₃, —NO₂, —OR⁶, —NR⁶R⁷ and C₁₋₆-alkyl,

R⁶ and R⁷ independently are hydrogen or C₁₋₆-alkyl,

or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

In still a further embodiment B is

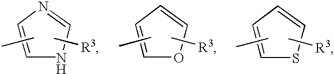

wherein R³ is

hydrogen, halogen, C₁₋₆-alkyl,

phenyl, which is substituted with one halogen substituent.

In another embodiment B is

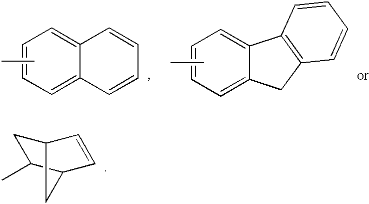

In a further embodiment Z is a valence bond, —CH₂—, —(CH₂)₂—, —(CH₂)₃—, —CH(CH₃)— or

In still a further embodiment D is

a valence bond, —CH₂—, —(CH₂)₂—, —(CH₂)₃—, —(CH₂)₄—, —(CH₂)₂—O— or

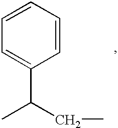

such as a valence bond, —CH₂— or —(CH₂)₂—O—, eg —CH₂— or —(CH₂)₂—O—.

In yet a further embodiment E is

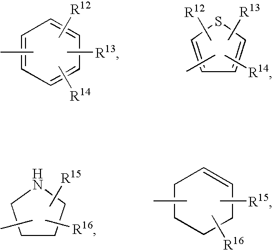

wherein R¹² to R¹⁶ are as defined for formula (I).

In one embodiment E is

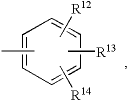

wherein R¹², R¹³ and R¹⁴ independently are hydrogen, halogen, —CF₃, —OCF₃, C₁₋₆-alkyl, C₃₋₈-cycloalkyl, C₄₋₈-cycloalkenyl or aryl.

In another embodiment E is

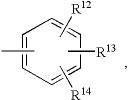

wherein R¹², R¹³ and R¹⁴ independently are hydrogen, halogen, —CF₃, —OCF₃ or C₁₋₆-alkyl.

In yet another embodiment E is

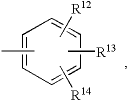

wherein R¹² is hydrogen, and R¹³ and R¹⁴ independently are halogen, —CF₃, —OCF₃ or C₁₋₆-alkyl.

In one embodiment R¹² is hydrogen, and R¹³ and R¹⁴ are both halogen or are both —CF₃.

In still another embodiment E is

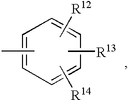

wherein R¹² and R¹³ are both hydrogen, and R¹⁴ is halogen, —CF₃, —OCF₃ or C₁₋₆-alkyl.

In yet another embodiment E is

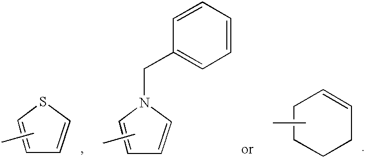

In yet a further embodiment X is —N═.

The compounds of the present invention may be chiral, and it is intended that any enantiomers, as separated, pure or partially purified enantiomers or racemic mixtures thereof are included within the scope of the invention.

Furthermore, when a double bond or a fully or partially saturated ring system or more than one center of asymmetry or a bond with restricted rotatability is present in the molecule diastereomers may be formed. It is intended that any diastereomers, as separated, pure or partially purified diastereomers or mixtures thereof are included within the scope of the invention.

Furthermore, some of the compounds of the present invention may exist in different tautomeric forms and it is intended that any tautomeric forms, which the compounds are able to form, are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methyl-, dimethyl-, trimethyl-, ethyl-, hydroxyethyl-, diethyl-, butyl-, tetramethylammonium salts and the like.

Also intended as pharmaceutically acceptable acid addition salts are the hydrates, which the present compounds, are able to form.

Furthermore, the pharmaceutically acceptable salts comprise basic amino acid salts such as lysine, arginine and ornithine.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming pharmacologically active substances. In general, such prodrugs will be functional derivatives of present compounds, which are readily convertible in vivo into the required compound. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The compounds according to the present invention act to antagonize the action of glucagon and are accordingly useful for the treatment of disorders and diseases in which such an antagonism is beneficial.

The compounds according to the invention preferably have an IC₅₀ value of no greater than 5 μM as determined by the Glucagon Binding Assay (I) or Glucagon Binding Assay (II) disclosed herein.

More preferably, the compounds according to the invention have an IC₅₀ value of less than 1 μM, preferably of less than 500 nM and even more preferred of less than 100 nM as determined by the Glucagon Binding Assay (I) or Glucagon Binding Assay (II) disclosed herein.

Furthermore, the compounds according to the invention preferably have a higher binding affinity to the glucagon receptor than to the GIP receptor.

Accordingly, the present compounds may be applicable for the treatment of hyperglycemia, IGT (impaired glucose tolerance), insulin resistance syndromes, syndrome X, type 1 diabetes, type 2 diabetes, hyperlipidemia, dyslipidemia, hypertriglyceridemia, hyperlipoproteinemia, hypercholesterolemia, arteriosclerosis including atherosclerosis, glucagonomas, acute pancreatitis, cardiovascular diseases, hypertension, cardiac hypertrophy, gastrointestinal disorders, obesity, diabetes as a consequence of obesity, diabetic dyslipidemia, etc.

Furthermore, they may be applicable as diagnostic agents for identifying patients having a defect in the glucagon receptor, as a therapy to increase gastric acid secretions and to reverse intestinal hypomobility due to glucagon administration.

They may also be useful as tool or reference molecules in labelled form in binding assays to identify new glucagon antagonists.

Accordingly, in a further aspect the invention relates to a compound according to the invention for use as a medicament.

The invention also relates to pharmaceutical compositions comprising, as an active ingredient, at least one compound according to the invention together with one or more pharmaceutically acceptable carriers or excipients.

The pharmaceutical composition is preferably in unit dosage form, comprising from about 0.05 mg to about 1000 mg, preferably from about 0.1 mg to about 500 mg and especially preferred from about 0.5 mg to about 200 mg of the compound according to the invention.

Furthermore, the invention relates to the use of a compound of the general formula (I′):

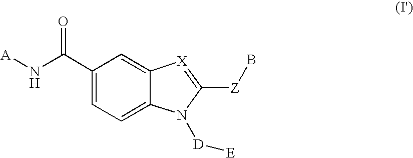

wherein

A is

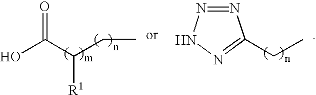

m is 0 or 1,

n is 0, 1, 2 or 3,

with the proviso that m and n must not both be 0,

R¹ is hydrogen, fluoro or sulfonic, —(CH₂)ₒ—OR²,

o is 0 or 1,

R² is hydrogen, C₁₋₆-alkyl, C₁₋₆-alkanoyl, aryl or aryl-C₁₋₆-alkyl,

X is —N═ or —CH═,

B is

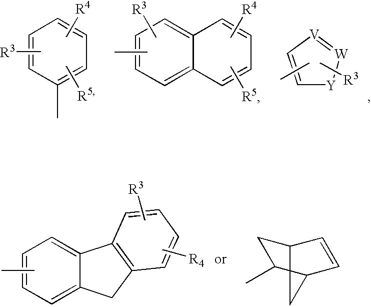

V and W independently are —CH═ or —N═,

Y is —O—, —S— or —NH—,

R³, R⁴ and R⁵ independently are

hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂R⁷, —S(O)₂NR⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,

C₁₋₆-alkyl, C₂₋₆-alkenyl or C₂₋₆-alkynyl,

which may optionally be substituted with one or more substituents selected from fluoro, —CN, —CF₃, —OCF₃, —OR⁶ and —NR⁶R⁷,

C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,

of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,

aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,

of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,

R⁶ and R⁷ independently are hydrogen or C₁₋₆-alkyl,

or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or two of the groups R³ to R⁵ when placed in adjacent positions together may form a bridge —(CR⁸R⁹)ₛ—O—(CR¹⁰R¹¹)ₜ—O—,

s is 0, 1 or 2,

t is 1 or 2,

R⁸, R⁹, R¹⁰ and R¹¹ independently are hydrogen, C₁₋₆-alkyl or fluoro,

D is —(CH₂)ₚ—,

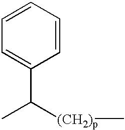

or —(CH₂)ₚ—O—,

p is 0, 1, 2, 3 or 4,

E is

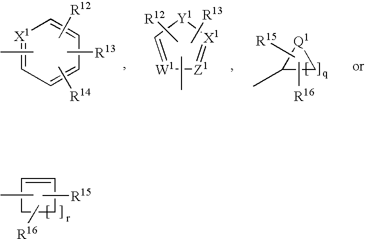

X¹, Z¹ and W¹ independently are —CH═ or —N═,

Y¹ is —O—, —S— or —NH—,

Q^{1 is —CH}₂— or —NH—,

q is 2, 3, 4, 5 or 6,

r is 1, 2, 3, 4 or 5,

R¹², R¹³ and R¹⁴ independently are

hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR⁷R¹⁸, —OCH₂C(O)NR¹⁷R¹⁸, —OC(O)R¹⁷, —C(O)R¹⁷ or —C(O)OR¹⁷,

C₁₋₆-alkyl, C₂₋₆-alkenyl or C₂₋₆-alkynyl,

which may optionally be substituted with one or more substituents selected from fluoro, —CN, —CF₃, —OCF₃, —OR¹⁷ and —NR¹⁷R¹⁸,

C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkly, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,

of which the cyclic moieties may optionally be substituted with one or more substituents selected from fluoro, —C(O)OR¹⁷, —CN, —CF₃, —OCF₃, —OR¹⁷ and —NR¹⁷R¹⁸,

aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,

of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,

R¹⁷ and R¹⁸ independently are hydrogen or C₁₋₆-alkyl,

or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or two of the groups R¹² to R¹⁴ when placed in adjacent positions together may form a bridge —(CR¹⁹R²⁰)ₓ—O—(CR²¹R²²)_{y}—O—,

x is 0, 1 or 2,

y is 1 or 2,

R¹⁹, R²⁰, R²¹ and R²² independently are hydrogen, C₁₋₆-alkyl or fluoro,

R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,

wherein the cyclic moieties may optionally be substituted with one or more substituents selected from halogen, —CN, —CF₃, —NO₂, —OR²³, —NR²³R²⁴ and C₁₋₆-alkyl,

R²³ and R²⁴ independently are hydrogen or C₁₋₆-alkyl, or

R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

or E is

C₁₋₆-alkyl, C₂₋₆-alkenyl or C₂₋₆-alkynyl,

which may optionally be substituted with one or more substituents selected from halogen, —CN, —CF₃, —OCF₃, —NO₂, —OR²⁵,—SR²⁵, —NR²⁵R²⁶ and C₁₋₆-alkyl,

R²⁵ and R²⁶ independently are hydrogen or C₁₋₆-alkyl, or

R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,

Z is —(CR²⁷R²⁸)ᵥ—(O)_{w}—(CR²⁹R³⁰)_{z}—,

v and z independently are 0, 1 or 2,

w is 0 or 1,

R²⁷, R²⁸, R²⁹ and R³⁰ independently are hydrogen or C₁₋₆-alkyl,

as well as any diastereomer or enantiomer or tautomeric form thereof including mixtures of these or a pharmaceutically acceptable salt thereof for the preparation of a medicament for the treatment of disorders or diseases, wherein a glucagon antagonistic action is beneficial.

The invention also relates to a method for the treatment of disorders or diseases, wherein a glucagon antagonistic action is beneficial the method comprising administering to a subject in need thereof an effective amount of a compound according to the invention.

In one embodiment of the invention the present compounds are used for the preparation of a medicament for the treatment of any glucagon-mediated conditions and diseases.

In another embodiment of the invention the present compounds are used for the preparation of a medicament for the treatment of hyperglycemia.

In yet another embodiment of the invention the present compounds are used for the preparation of a medicament for lowering blood glucose in a mammal. The present compounds are effective in lowering the blood glucose, both in the fasting and the postprandial stage.

In still another embodiment of the invention the present compounds are used for the preparation of a pharmaceutical composition for the treatment of IGT.

In a further embodiment of the invention the present compounds are used for the preparation of a pharmaceutical composition for the treatment of type 2 diabetes.

In yet a further embodiment of the invention the present compounds are used for the preparation of a pharmaceutical composition for the delaying or prevention of the progression from IGT to type 2 diabetes.

In yet another embodiment of the invention the present compounds are used for the preparation of a pharmaceutical composition for the delaying or prevention of the progression from non-insulin requiring type 2 diabetes to insulin requiring type 2 diabetes.

In a further embodiment of the invention the present compounds are used for the preparation of a pharmaceutical composition for the treatment of type 1 diabetes. Such treatment is normally accompanied by insulin therapy.

In yet a further embodiment of the invention the present compounds are used for the preparation of a pharmaceutical composition for the treatment of obesity.

In still a further embodiment of the invention the present compounds are used for the preparation of a pharmaceutical composition for the treatment of disorders of the lipid metabolism.

In still another embodiment of the invention the present compounds are used for the preparation of a pharmaceutical composition for the treatment of an appetite regulation or energy expenditure disorder.

In a further embodiment of the invention, treatment of a patient with the present compounds is combined with diet and/or exercise.

In a further aspect of the invention the present compounds are administered in combination with one or more further active substances in any suitable ratios. Such further active substances may eg be selected from antidiabetics, antiobesity agents, antihypertensive agents, agents for the treatment of complications resulting from or associated with diabetes and agents for the treatment of complications and disorders resulting from or associated with obesity.

Thus, in a further embodiment of the invention the present compounds may be administered in combination with one or more antidiabetics.

Suitable antidiabetic agents include insulin, insulin analogues and derivatives such as those disclosed in EP 792 290 (Novo Nordisk A/S), eg N^{εB29}-tetradecanoyl des (B30) human insulin, EP 214 826 and EP 705 275 (Novo Nordisk A/S), eg Asp^{B28} human insulin, U.S. Pat. No. 5,504,188 (Eli Lilly), eg LyS^{B28} Pro^{B29} human insulin, EP 368 187 (Aventis), eg Lantus®, which are all incorporated herein by reference, GLP-1 and GLP-1 derivatives such as those disclosed in WO 98/08871 (Novo Nordisk A/S), which is incorporated herein by reference, as well as orally active hypoglycemic agents.

The orally active hypoglycemic agents preferably comprise imidazolines, sulphonylureas, biguanides, meglitinides, oxadiazolidinediones, thiazolidinediones, insulin sensitizers, insulin secretagogues, such as glimepride, α-glucosidase inhibitors, agents acting on the ATP-dependent potassium channel of the β-cells eg potassium channel openers such as those disclosed in WO 97/26265, WO 99/03861 and WO 00/37474 (Novo Nordisk A/S) which are incorporated herein by reference, or mitiglinide, or a potassium channel blocker, such as BTS-67582, nateglinide, glucagon antagonists such as those disclosed in WO 99/01423 and WO 00/39088 (Novo Nordisk A/S and Agouron Pharmaceuticals, Inc.), which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antilipidemic agents, compounds lowering food intake, PPAR (peroxisome proliferator-activated receptor) and R×R (retinoid×receptor) agonists, such as ALRT-268, LG-1268 or LG-1069.

In one embodiment, the present compounds are administered in combination with insulin or an insulin analogue or derivative, such as N^{εB29}-tetradecanoyl des (B30) human insulin, Asp^{B28} human insulin, Lys^{B28} Pro^{B29} human insulin, Lantus®, or a mix-preparation comprising one or more of these.

In a further embodiment of the invention the present compounds are administered in combination with a sulphonylurea eg tolbutamide, chlorpropamide, tolazamide, glibenclamide, glipizide, glimepiride, glicazide or glyburide.

In another embodiment of the invention the present compounds are administered in combination with a biguanide eg metformin.

In yet another embodiment of the invention the present compounds are administered in combination with a meglitinide eg repaglinide or nateglinide.

In still another embodiment of the invention the present compounds are administered in combination with a thiazolidinedione insulin sensitizer eg troglitazone, ciglitazone, pioglitazone, rosiglitazone, isaglitazone, darglitazone, englitazone, CS-011/CI-1037 or T 174 or the compounds disclosed in WO 97/41097, WO 97/41119, WO 97/41120, WO 00/41121 and WO 98/45292 (Dr. Reddy's Research Foundation), which are incorporated herein by reference.

In still another embodiment of the invention the present compounds may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In a further embodiment of the invention the present compounds are administered in combination with an α-glucosidase inhibitor eg voglibose, emiglitate, miglitol or acarbose.

In another embodiment of the invention the present compounds are administered in combination with an agent acting on the ATP-dependent potassium channel of the β-cells eg tolbutamide, glibenclamide, glipizide, glicazide, BTS-67582 or repaglinide.

In yet another embodiment of the invention the present compounds may be administered in combination with nateglinide.

In still another embodiment of the invention the present compounds are administered in combination with an antilipidemic agent eg cholestyramine, colestipol, clofibrate, gemfibrozil, lovastatin, pravastatin, simvastatin, probucol or dextrothyroxine.

In another embodiment of the invention, the present compounds are administered in combination with more than one of the above-mentioned compounds eg in combination with metformin and a sulphonylurea such as glyburide; a sulphonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In a further embodiment of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, R×R (retinoid×receptor) modulators, TR β agonists, AGRP (Agouti related protein) inhibitors, ′H3 histamine antagonists, opioid antagonists (such as naltrexone), exendin-4, GLP-1 and ciliary neurotrophic factor.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

In still another embodiment the antiobesity agent is phendimetrazine, diethylpropion, fluoxetine, bupropion, topiramate or ecopipam.

Furthermore, the present compounds may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds according to the invention with diet and/or exercise, one or more of the above-mentioned compounds and optionally one or more other active substances are considered to be within the scope of the present invention.

Pharmaceutical Compositions

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typical doses are in the order of about half the dose employed for oral administration.

The compounds of this invention are generally utilized as the free substance or as a pharmaceutically acceptable salt thereof. One example is a base addition salt of a compound having the utility of a free acid. When a compound of the formula (I) contains a free acid such salts are prepared in a conventional manner by treating a solution or suspension of a free acid of the formula (I) with a chemical equivalent of a pharmaceutically acceptable base. Representative examples are mentioned above.

For parenteral administration, solutions of the novel compounds of the formula (I) in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the novel compounds of the formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablelted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet that may be prepared by conventional tabletting techniques may contain:

| Core: | | | |
|---|---|---|---|
| Active compound (as free | | 5.0 | mg |
| compound or salt thereof) | | | |
| Lactosum Ph. Eur. | | 67.8 | mg |
| Cellulose, microcryst. (Avicel) | | 31.4 | mg |
| Amberlite ® IRP88* | | 1.0 | mg |
| Magnesii stearas Ph. Eur. | | q.s. | |
| Coating: | | | |
| Hydroxypropyl methylcellulose | approx. | 9 | mg |
| Mywacett 9-40 T** | approx. | 0.9 | mg |

*Polacrillin potassium NF, tablet disintegrant, Rohm and Haas.
**Acylated monoglyceride used as plasticizer for film coating.

If desired, the pharmaceutical composition of the invention may comprise the compound of the formula (I) in combination with further pharmacologically active substances such as those described in the foregoing.

### EXAMPLES

The following examples and general procedures refer to intermediate compounds and final products identified in the specification and in the synthesis schemes. The preparation of the compounds of the present invention is described in detail using the following examples, but the chemical reactions described are disclosed in terms of their general applicability to the preparation of the glucagon antagonists of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials. All temperatures are set forth in degrees Celsius and unless otherwise indicated, all parts and percentages are by weight when referring to yields and all parts are by volume when referring to solvents and eluents.

Some of the NMR data shown in the following examples are only selected data.

In the examples the following terms are intended to have the following, general meanings:

| | |
|---|---|
| BSA: | N,O-bis(trimethylsilyl)acetimidate |
| DCE: | 1,2-dichloroethane |
| DCM: | dichloromethane, methylenechloride |
| DIC: | dilsopropylcarbodiimide |
| DIPEA: | diisopropylethylamine |
| DMSO: | dimethyl sulphoxide |
| Fmoc: | 9-fluorenylmethyloxycarbonyl |
| HOBt: | 1-hydroxybenzotriazole |
| MeOH: | methanol |
| NMP: | N-methyl-2-pyrrolidinone |
| TEA: | trifluoroacetic acid |

HPLC-MS (Method A)

The following instrumentation was used:

Sciex APl 100 Single quadropole mass spectrometer

Perkin Elmer Series 200 Quard pump

Perkin Elmer Series 200 autosampler

Applied Biosystems 785A UV detector

Sedex 55 evaporative light scattering detector

A Valco column switch with a Valco actuator controlled by timed events from the pump.

The Sciex Sample control software running on a Macintosh PowerPC 7200 computer was used for the instrument control and data acquisition.

The HPLC pump was connected to four eluent reservoirs containing:

| | |
|---|---|
| A: | Acetonitrile |
| B: | Water |
| C: | 0.5% TFA in water |
| D: | 0.02 M ammonium acetate |

The requirements for the samples are that they contain approximately 500 μg/ml of the compound to be analysed in an acceptable solvent such as methanol, ethanol, acetonitrile, THF, water and mixtures thereof. (High concentrations of strongly eluting solvents will interfere with the chromatography at low acetonitrile concentrations.)

The analysis was performed at room temperature by injecting 20 μl of the sample solution on the column, which was eluted with a gradient of acetonitrile in either 0.05% TFA or 0.002 M ammonium acetate. Depending on the analysis method varying elution conditions were used.

The eluate from the column was passed through a flow splitting T-connector, which passed approximately 20 μl /min through approx. 1 m 75μ fused silica capillary to the APl interface of APl 100 spectrometer.

The remaining 1.48 ml/min was passed through the UV detector and to the ELS detector.

During the LC-analysis the detection data were acquired concurrently from the mass spectrometer, the UV detector and the ELS detector.

The LC conditions, detector settings and mass spectrometer settings used for the different methods are given in the following table.

| | | | | |
|---|---|---|---|---|
| Column | YMC ODS-A 120Å s − 5μ 3 mm × 50 mm id | | | |
| Gradient | 5%-90% acetonitrile in 0.05% TFA linearly during 7.5 min at 1.5 ml/min | | | |
| Detection | UV: 214 nm | ELS: 40° C. | | |
| MS | Experiment: | Start: 100 amu | Stop: 800 amu | Step: 0.2 amu |
| | Dwell: | 0.571 msec | | |
| | Method: | Scan 284 times = 9.5 min | | |

General Procedure (A)

The compounds of formula (I₁) can be prepared on solid support using the following procedure:

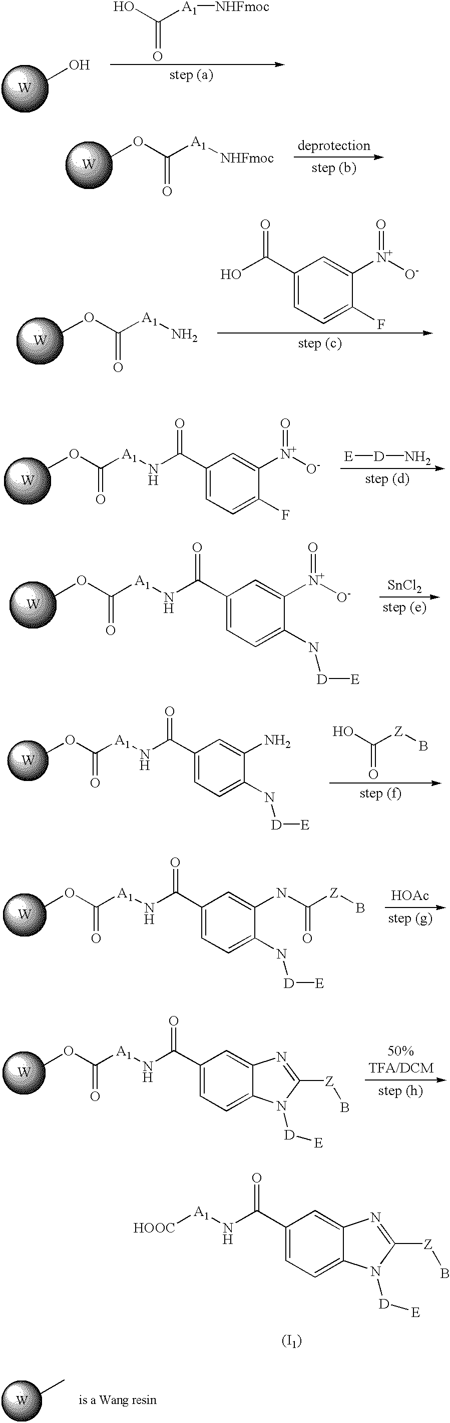

wherein

A₁ is

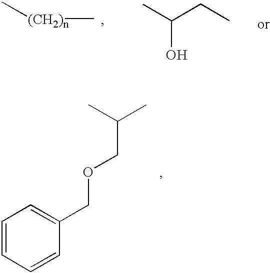

and n, D, E, Z and B are as defined for formula (I).

Attachment of an Fmoc-amino acid to the solid support (Wang resin) is performed in step (a). This can be done using either in situ generation of a symmetric anhydride with a carbodiimide or activation to an active ester such as HOBt ester. In step (b) the Fmoc-group is deprotected eg by using piperidine as base. Then in step (c) the resin-bound amine is acylated with 4-fluoro-3-nitrobenzoic by eg in situ generation of a symmetric anhydride with a carbodiimide. In the following step (d) an aromatic nucleophilic substitution with an amine is carried out in an aprotic solvent such as DMSO. In step (e) the nitro group is reduced using stannous chloride dihydrate, and the amine product is subsequently acylated with a carboxylic acid activated either as its HOBt ester or by its symmetric anhydride (step (f)). When A₁ is —CHOHCH₂-step (f) is performed using 1) BSA and 2) B—Z—COOH. Otherwise, step (f) is performed using only B—Z—COOH. The acylated product is then cyclised to a benzimidazole using acetic acid catalysis (step (g)). Cleavage of the final compound (I₁) is achieved in step (h) by use of an acid (eg TFA in DCM).

Protocol for Synthesis of Compounds of Formula (I₁) According to General Procedure (A) (examples 1 to 25):

Step (a): Attachment of Fmoc-amino Acid to Wang Resin

1 ml 0.55 M N-Fmoc-amino acid is activated with 0.29 M DIC (0.45 ml) for at least 10 min and the solution is added to the resin. A solution of a 0.05 M 4-dimethylaminopyridine in NMP (0.1 ml) is added and the mixture is shaken at room temperature for 15 hours. The excess of reagents is removed. The resin is washed with NMP (4×1 ml).

Step (b): Removal of the Fmoc-Protecting Group

1000 μl of a 20% piperidine in NMP is transferred to the Fmoc-protected resin. The mixture is shaken for 10 min at room temperature. The well is emptied and the procedure is repeated. The resin is washed with NMP (6×1 ml).

Step (c): Acvlation with 4-fluoro-3-nitrobenzoic Acid

1000 μl of a 0.8 M 4-fluoro-3-nitrobenzoic acid solution in NMP/DCE (1:1 (v/v)) is added to the resin followed by 0.2 ml of a 2 M DIC solution in DCE. The mixing should be started immediately after the DIC addition. The mixture is shaken for 12 hours at room temperature. The well is emptied and the resin is washed with NMP (5×1.5 ml).

Step (d): Nucleophilic Aromatic Substitution of Aromatic Fluoride with an Amine

1500 μl of a 0.9 M amine solution in DMSO is transferred to the resin. The reaction is run at room temperature for 9.5 hours at 450 rpm. The well is emptied and washed once with NMP (1×1500 μl) and then with DCM (3×1500 μl).

Step (e): Reduction of NO₂-group with Stannous Chloride Dihydrate

To the resin, is added 1250 μl freshly prepared stannous chloride dihydrate (240 mg) solution in NMP. The resin is shaken at 450 rpm for 12 hours. The resin is drained and washed with NMP (5×1.5 ml).

Step (f): Acylation with B—Z—COOH

A 0.5 M solution of carboxylic acid in NMP (1 ml) is added 0.25 mmol DIC, and the mixture is added to the resin. The reaction is run at room temperature for 16 hours at 450 rpm. The resin is drained and washed with NMP (5×1.5 ml).

Step (g): Benzimidazole Formation

Acetic acid glacial (1500 μl) is added to the resin. The resin is shaken at 450 rpm for 72 hours at 80° C. The resin is then washed with NMP (3×2 ml) followed by DCM (10×2 ml).

Step (h): Cleavage

A TFA:DCM solution (50:50, 1200 μl) is added to the resin. The mixture is shaken and left for one hour. The well is emptied for 5 min into a cleavage vial and concentrated in vacuo to afford the product of formula (I₁).

### Example 1

(General Procedure (A))

3-{[1-(3,5-Dichlorobenzyl)-2-(3,5-dimethoxyphenyl)-1H-benzimidazole-5-carbonyl]amino}-propionic acid

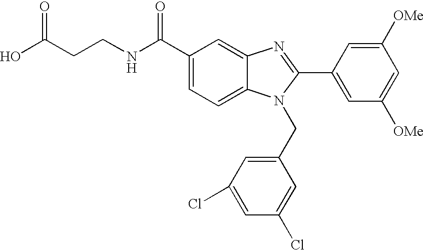

¹H NMR (DMSO): δ8.55 (t, 1H); 8.26 (s, 1H); 7.82 (d, 1H); 7.63 (d, 1H); 7.50 (s, 1H); 7.05 (s, 2H); 6.80 (s, 2H); 6.68 (s, 1H); 5.64 (s, 2H).

### Example 2

(General Procedure (A)

3-{[1-(3,5-Dichlorobenzyl)-2-(3-trifluoromethoxyphenyl)-1H-benzimidazole-5-carbonyl]amino}-propionic acid))

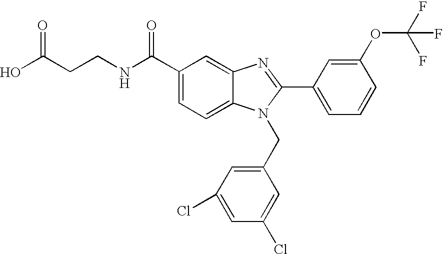

¹H NMR (DMSO): δ8.55 (t, 1H); 8.17 (s, 1H); 7.81 (d, 1H); 7.63-7.78 (m, 4H); 7.53 (d, 1H); 7.46 (s, 1H); 6.95 (s, 2H); 5.65 (s, 2H).

### Example 3

(General Procedure (A))

3-{[1-(3,5-Dichlorobenzyl)-2-(4-nitrophenyl)-1H-benzimidazole-5-carbonyl]amino}propionic acid

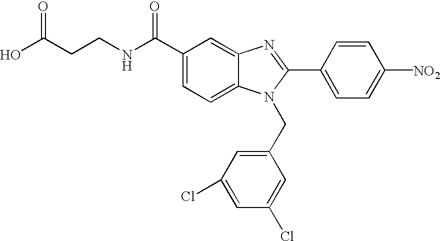

¹H NMR (DMSO): δ8.57 (t, 1H); 8.39 (d, 2H); 8.29 (s, 1H); 8.01 (d, 2H); 7.85 (d, 1H); 7.65 (d, 1H); 7.50 (s, 1H); 6.98 (s, 2H); 5.72 (s, 2H).

### Example 4

(General Procedure (A))

3-{[1-(3,5-Dichlorobenzyl)-2-(2-o-tolylethyl)-1H-benzimidazole-5-carbonyl]amino}propionic acid

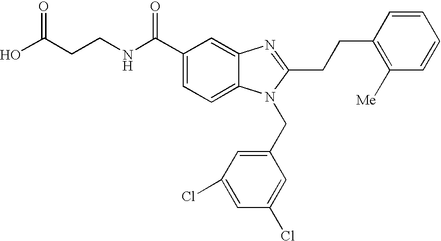

¹H NMR (DMSO): δ8.48 (t, 1H); 8.16 (s, 1H); 7.74 (d, 1H); 7.53 (d, 2H); 6.99-7.21 (m, 6H); 5.58 (s, 2H).

### Example 5

(General Procedure (A)) 3-{[1-(3,5-Dichlorobenzyl)-2-(2-p-tolylethyl)-1H-benzimidazole-5-carbonyl]amino}propionic acid

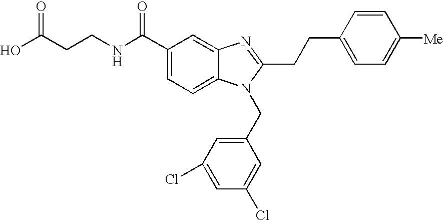

¹H NMR (DMSO): δ8.45 (t, 1H); 8.11 (s, 1H); 7.74 (d, 1H); 7.52 (d, 2H); 7.09 (d, 2H); 6.98 (dd, 4H); 5.51 (s, 2H).

### Example 6

(General Procedure (A))

3-{[1-(3,5-Dichlorobenzyl)-2-(1-phenoxyethyl)-1H-benzimidazole-5-carbonyl]amino}propionic acid

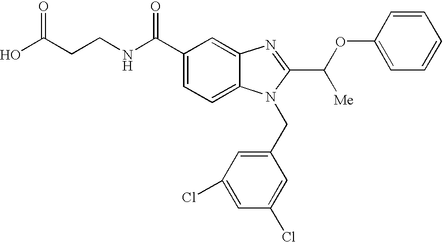

¹H NMR (DMSO): δ8.52 (t, 1H); 8.22 (s, 1H); 7.75 (d, 1H); 7.51 (d, 1H); 7.42 (s, 1H); 7.19 (m, 2H); 6.91 (m, 3H); 6.82 (d, 2H); 5.95 (dd, 1H); 5.62 (dd, 2H).

### Example 7

(General Procedure (A))

3-{[2-(3-Trifluoromethoxyphenyl)-1-(3-trifluoromethylbenzyl)-1H-benzimidazole-5-carbonyl]-amino}propionic acid

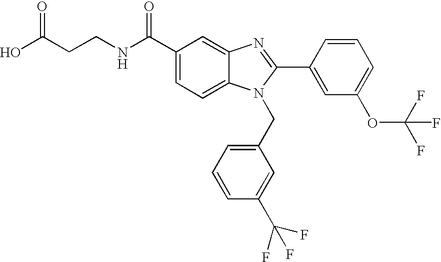

¹H NMR (DMSO): δ8.56 (t, 1H); 8.28 (s, 1H); 7.85 (d, 1H); 7.78 (d, 1H); 7.71 (m, 2H); 7.61 (m, 2H); 7.43-7.58 (m, 2H); 7.34 (s, 1H); 7.18 (d, 1H); 5.75 (s, 2H).

### Example 8

(General Procedure (A))

3-{[2-Naphthalen-2-ylmethyl-1-(3-trifluoromethylbenzyl)-1H-benzimidazole-5-carbonyl]-amino}propionic acid

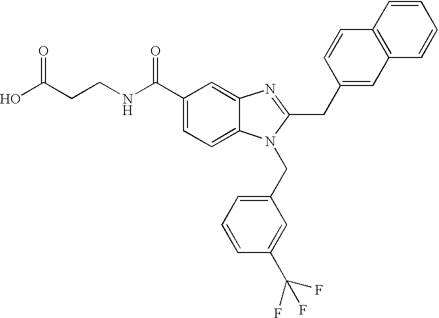

¹H NMR (DMSO): δ8.49 (t, 1H); 8.18 (s, 1H); 7.65-7.85 (m, 5H); 7.32-7.53 (m, 6H); 7.24 (s, 1H); 7.16 (d, 1H); 5.72 (s, 2H); 4.52 (s, 2H).

### Example 9

(General Procedure (A))

3-{[2-(2-Chlorobenzyl)-1-(2-phenoxyethyl)-1H-benzimidazole-5-carbonyl]amino}propionic acid

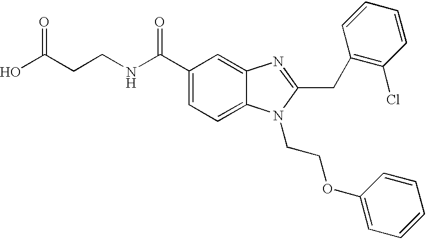

¹H NMR (DMSO): δ8.43 (t, 1H); 8.05 (s, 1H); 7.80 (d, 1H); 7.70 (d, 1H); 7.50 (s, 1H); 7.19-7.41 (m, 3H); 7.26 (m, 2H); 6.95 (t, 1H); 6.84 (d, 2H); 4.70 (d, 2H); 4.53 (s, 2H); 4.28 (d, 2H).

### Example 10

(General Procedure (A))

3-{[1-(2-Phenoxyethyl)-2-(4-trifluoromethoxyphenyl)-1H-benzimidazole-5-carbonyl]amino}-propionic acid

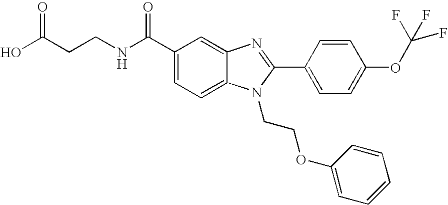

¹H NMR (DMSO): δ8.53 (t, 1H); 8.21 (s, 1H); 7.99 (d, 2H); 7.85 (s, 2H); 7.57 (d, 2H); 7.19 (dd, 2H); 6.89 (t, 1H); 6.70 (d, 2H); 4.73 (d, 2H); 4.29 (d, 2H).

### Example 11

(General Procedure (A))

3-{[2-(3,5-Dimethoxyphenyl)-1-(4-phenylbutyl)-1H-benzimidazole-5-carbonyl]amino}propionic acid

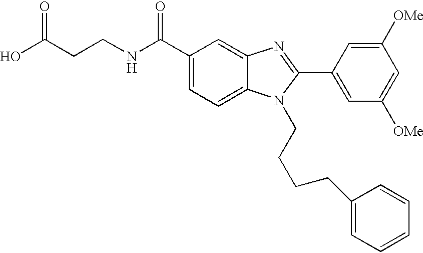

¹H NMR (DMSO): δ8.54 (s, 1H); 8.21 (d, 1H); 7.84 (d, 1H); 7.12-7.29 (m, 5H); 7.05 (d, 2H); 6.88 (s, 2H); 6.72 (s, 1H); 4.38 (m, 2H).

### Example 12

(General Procedure (A))

3-{[1-(4-Phenylbutyl)-2-(3-trifluoromethoxyphenyl)-1H-benzimidazole-5-carbonyl]amino}-propionic acid

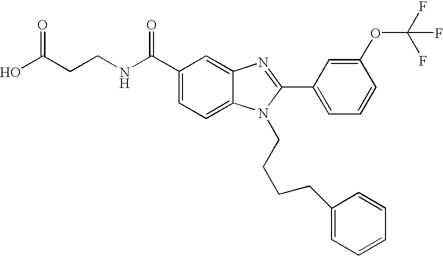

¹H NMR (DMSO): δ8.53 (t, 1H); 8.22 (s, 1H); 7.07-7.37 (m, 3); 7.03 (d, 2H); 6.69-6.91 (m, 5H); 5.60 (d, 1H); 4.39 (t, 2H).

### Example 13

(General Procedure (A))

3-{[2-Naphthalen-2-ylmethyl-1-(4-phenylbutyl)-1H-benzimidazole-5-carbonyl]amino}propionic acid

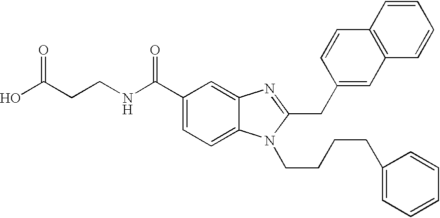

¹H NMR (DMSO): δ8.50 (t, 1H); 8.12 (s, 1H); 7.99 (d, 2H); 7.81-7.97 (m, 5H); 7.78 (d, 1H); 7.61 (d, 1H); 7.48 (m, 2H); 7.43 (d, 1H); 7.07-7.23 (m, 3H); 4.55 (s, 2H); 4.28 (m, 2H).

### Example 14

(General Procedure (A))

3-{[1-(4-Phenylbutyl)-2-(1-phenylethyl)-1H-benzimidazole-5-carbonyl]amino}propionic acid

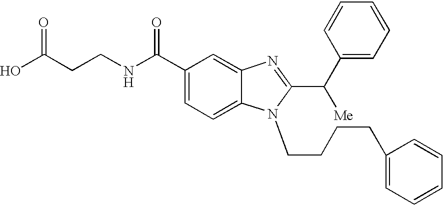

¹H NMR (DMSO): δ8.48 (t, 1H); 8.18 (s, 1H); 7.77 (d, 1H); 7.55 (d, 1H); 7.14-7.37 (m, 8H); 7.12 (d, 2H); 4.62 (dd, 1H); 4.18 (m, 1H); 4.02 (m, 1H).

### Example 15

(General Procedure (A))

3-{[1-(4-Phenylbutyl)-2-(3-phenylpropyl)-1H-benzimidazole-5-carbonyl]amino}propionic acid

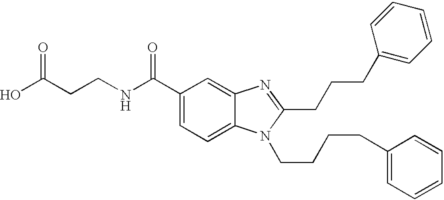

¹H NMR (DMSO): δ8.62 (t, 1H); 8.13 (s, 1H); 7.95 (d, 1H); 7.75 (d, 1H); 7.15-7.35 (m, 10H); 4.31 (m, 2H).

### Example 16

(General Procedure (A))

3-{[1-(4-Phenylbutyl)-2-(3-thiophen-2-ylpropyl)-1H-benzimidazole-5-carbonyl]amino}propionic acid

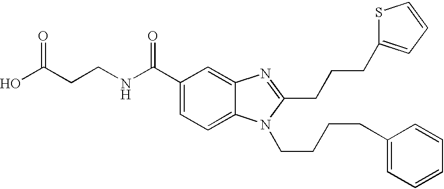

¹H NMR (DMSO): δ8.59 (t, 1H); 8.13 (s, 1H); 7.86 (d, 1H); 7.75 (d, 1H); 7.33 (d, 1H); 7.12-7.31 (m, 5H); 6.96 (m, 1H); 6.91 (s, 1H); 4.31 (m, 2H).

### Example 17

(General Procedure (A))

3-{[1-(4-Phenylbutyl)-2-(2-trifluoromethyl-benzyl)-1H-benzimidazole-5-carbonyl]amino}-propionic acid

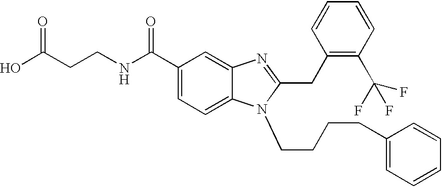

¹H NMR (DMSO): δ8.45 (t, 1H); 8.06 (s, 1H); 7.78 (t, 2H); 7.63 (t, 2H); 7.51 (t, 1H); 7.06-7.36 (m, 6H); 4.48 (s, 2H); 4.25 (t, 2H).

### Example 18

(General Procedure (A))

3-{[2-[2-(2-Methoxyphenyl)ethyl]-1-(4-phenylbutyl)-1H-benzimidazole-5-carbonyl]amino}-propionic acid

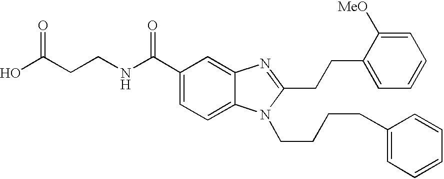

¹H NMR (DMSO): δ8.62 (t, 1H); 8.13 (s, 1H); 7.93 (d, 1H); 7.78 (d, 1H); 7.09-7.30 (m, 8H); 6.97 (d, 1H); 6.88 (t, 1H); 4.28 (t, 2H); 3.68 (s, 3H).

### Example 19

(General Procedure (A))

3-{[1-(4-Phenylbutyl)-2-thiophen-3-ylmethyl-1H-benzimidazole-5-carbonyl]amino}propionic acid

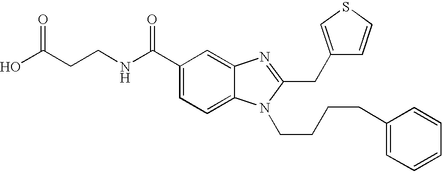

¹H NMR (DMSO): δ8.54 (t, 1H); 8.14 (s, 1H); 7.80 (d, 1H); 7.68 (d, 1H); 7.50 (m, 1H); 7.38 (s, 1H); 7.28 (m, 2H); 7.15 (m, 3H); 7.04 (d, 2H); 4.45 (s, 2H); 4.31 (m, 2H).

### Example 20

(General Procedure (A))

3-{[2-(3-Dimethylaminophenyl)-1-(4-phenylbutyl)-1H-benzimidazole-5-carbonyl]amino}-propionic acid

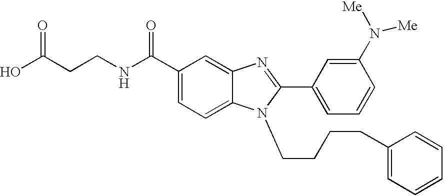

¹H NMR (DMSO): δ8.51 (t, 1H); 8.22 (s, 1H); 7.89 (d, 1H); 7.81 (d, 1H); 7.42 (t, 1H); 7.11-7.27 (m, 4H); 6.94-7.08 (m, 4H); 4.40 (t, 1H).

### Example 21

(General Procedure (A))

3-{[2-(4-Dimethylaminobenzyl)-1-(4-phenylbutyl)-1H-benzimidazole-5-carbonyl]amino}-propionic acid

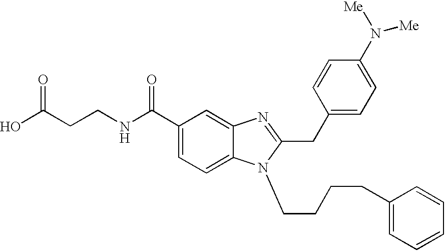

¹H NMR (DMSO): δ8.66 (t, 1H); 8.16 (s, 1H); 7.95 (d, 1H); 7.85 (d, 1H); 7.08-7.32 (m, 8H); 6.78 (d, 2H); 4.48 (s, 2H); 4.30 (m, 2H).

### Example 22

(General Procedure (A))

3-{[1-(3,5-Bis(trifluoromethyl)benzyl)-2-(3,5-dimethoxyphenyl)-1H-benzimidazole-5-carbonyl]-amino}propionic acid

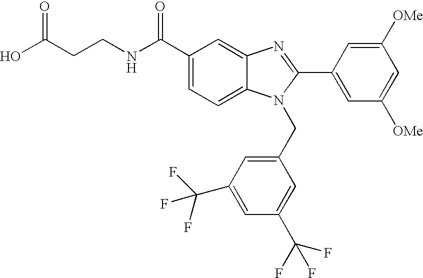

¹H NMR (DMSO): δ8.56 (t, 1H); 8.25 (s, 1H); 8.00 (s, 1H); 7.86 (d, 1H); 7.75 (d, 1H); 7.66 (s, 2H); 6.79 (s, 2H); 6.68 (s, 1H); 5.79 (s, 2H).

### Example 23

(General Procedure (A))

3-{[1-(3,5-Bis(trifluoromethyl)benzyl)-2-(1-phenylethyl)-1H-benzimidazole-5-carbonyl]amino}-propionic acid

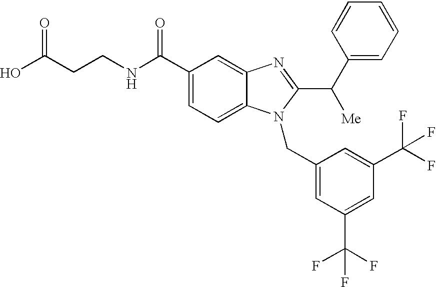

¹H NMR (DMSO): δ8.50 (t, 1H); 8.25 (s, 1H); 7.84 (s, 1H); 7.72 (d, 1H); 7.45 (d, 1H); 7.20-7.38 (m, 4H); 6.93-7.10 (m, 3H), 5.72 (dd, 2H); 4.67 (q, 1H).

9025-0115-0060

### Example 24

(General Procedure (A))

3-{[1-(3,5-Bis(trifluoromethyl)benzyl)-2-(1-phenoxyethyl)-1H-benzimidazole-5-carbonyl]-amino}propionic acid

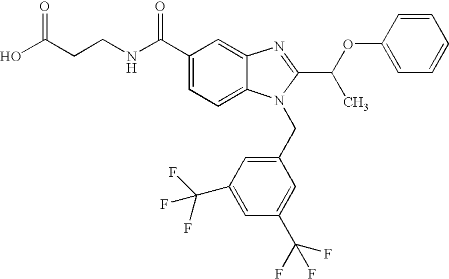

¹H NMR (DMSO): δ8.52 (t, 1H); 8.24 (s, 1H); 7.89 (s, 1H); 7.76 (d, 1H); 7.52 (m, 3H); 7.12 (m, 2H); 6.85 (t, 1H); 6.70 (d, 2H); 5.99 (q, 1H); 5.82 (dd, 2H).

### Example 25

(General Procedure (A))

3-{[1-(3,5-Bis(trifluoromethyl)benzyl)-2-(2-methoxybenzyl)-1H-benzimidazole-5-carbonyl]-amino}propionic acid

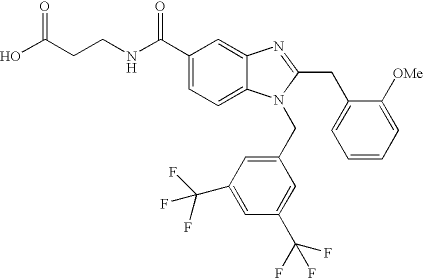

¹H NMR (DMSO): δ8.50 (t, 1H); 8.16 (s, 1H); 7.94 (s, 1H); 7.76 (d, 1H); 7.56 (d, 1H); 7.48 (s, 2H); 7.09 (m, 2H); 6.78 (m, 2H); 5.75 (s, 2H); 4.30 (s, 2H).

### General Procedure (B)

The compounds of formula (I₁) can also be prepared on solid support using the following procedure:

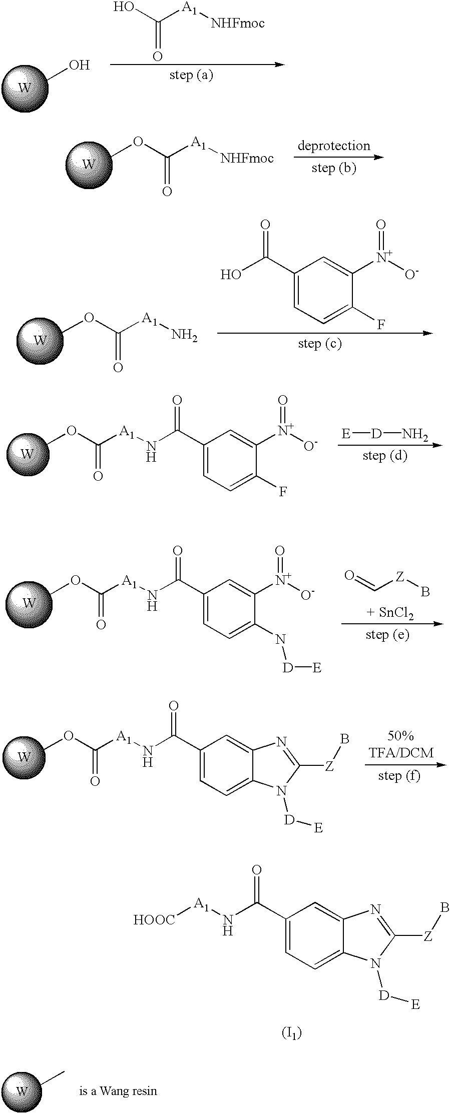

wherein

A₁ is

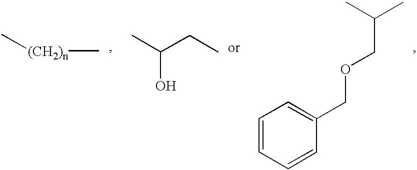

and n, Z, B, D and E are as defined for formula (I)

Attachment of an Fmoc-amino acid to the solid support (Wang resin) is performed in step (a). This can be done using either in situ generation of a symmetric anhydride with a carbodiimide or activation to an active ester such as eg HOBt ester. In step (b) the Fmoc-group is deprotected eg by using piperidine as base. Then in step (c) the resin-bound amine is acylated with 4-fluoro-3-nitrobenzoic by eg in situ generation of a symmetric anhydride with a carbodiimide. In the following step (d) an aromatic nucleophilic substitution with an amine is carried out in an aprotic solvent such as DMSO. The benzimidazole formation in step (e) can be performed as a one-pot reaction by adding an aldehyde together with a reducing reagent (eg stannous chloride dihydrate). Cleavage of the final compound of formula (I₁) in step (f) can be performed under acidic conditions using eg TFA in DCM.

Protocol for Synthesis of Compounds of Formula (I₁) (Examples 26 to 27):

Step (a): Altachment of Fmoc-amino Acid to Wang Resin

1 ml 0.55 M N-Fmoc-amino acid in NMP is activated with 0.29M DIC (0.45 ml) in toluene for at least 10 min and the solution is added to the resin. 0.05 M solution of 4-dimethylaminopyridine in NMP (0.1 ml) is added and the mixture is shaken at room temperature for 15 hours. The excess of reagents is removed by filtration and the resin is washed with NMP (4×1 ml).

Step (b): Removal of the Fmoc-protecting Group

1000 μl of a 20% piperidine in NMP is transferred to the Fmoc-protected resin. The mixture is shaken for 10 min at room temperature. The well is emptied and the procedure is repeated. The resin is washed with NMP (6×1 ml).

Step (c): Acylation with 4-fluoro-3-nitrobenzoic acid

1000 μl of a 0.8 M 4-fluoro-3-nitrobenzoic acid solution in NMP/DCE (1:1 (v/v)) is added to the resin followed by 0.2 ml of a 2 M DIC solution in DCE. The mixing should be started immediately after the DIC addition. The mixture is shaken for 12 hours at room temperature. The well is emptied and the resin is washed with NMP (5×1.5 ml).

Step (d): Nucleophilic Aromatic Substitution of Aromatic Fluoride with an Amine

1500 μl of a 0.9 M amine solution in DMSO is transferred to the resin containing well. The reaction is run at room temperature for 9.5 hours at 450 rpm. The well is emptied and washed once with NMP (1×1500 μl) and then with DCM (3×1500 μl).

Step (e): Benzimidazole Formation

1 ml of 0.5 M aldehyde solution in NMP is added followed by 2 ml of a fresh 1.1 M solution of stannous chloride dihydrate in NMP. The resulting mixture is shaken at room temperature under exclusion of air for 15 hours. The resin is drained and washed with NMP (4×1 ml).

Step (f): Cleavage

1200 μl TFA solution is added to the resin. Mix and wait for one hour. The well is emptied for 5 min into a cleavage vial and concentrated in vacuo.

The solid phase chemistry according to General Procedure (B) above was used for synthesis of libraries on semi automated and fully automated equipment. One 334 membered non-combinatorial library was produced for validation of the chemistry with the following result: 48 randomly chosen samples out of 334 compounds were analysed by HPLC-MS. All Fmoc-amino acids were represented at least two times. The expected mass was found in 85% of the samples. 65% of the samples had a purity of at least 50% and 25% were above 80% purity (see table below).

| | | | |
|---|---|---|---|
| Purity (%) | >80 | >50 | >10 |
| Number of samples (%) | 25 | 65 | 71 |

A second 346 membered non-combinatorial library was produced with the following results: 40 randomly chosen samples out of the 346 compounds were analysed on HPLC-MS. All Fmoc-amino acids were represented at least two times. The expected mass was found in 87% of the samples. 72% of the samples had a purity of at least 40% and 15% were above 80% purity (see table below).

| | | | |
|---|---|---|---|
| Purity (%) | >80 | >50 | >40 |
| Number of samples (%) | 15 | 59 | 72 |

A third 346 membered non-combinatorial library was produced with the following results: 39 randomly chosen samples out of the 346 compounds were analysed on HPLC-MS. The expected mass was found in 85% of the samples. 74% of the samples had a purity of at least 50% and 23% were above 80% purity (see table below).

| | | | |
|---|---|---|---|
| Purity in % | >80 | >50 | >10 |
| Number of samples % | 23 | 74 | 85 |

The following two compounds were found as hits (defined as compounds that show at least 40% displacement of radiotracer at 1 μM compound concentration) in Glucagon Binding Assay (II)) and resynthesised as single compounds:

### Example 26

3-({1-(3,5-Bis(trifluoromethyl)benzyl)-2-[3-(3-trifluoromethylphenoxy)phenyl]-1H-benzimid -azole-5-carbonyl}amino)propionic acid

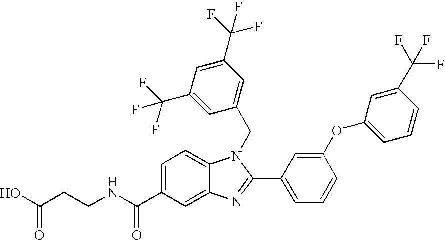

40.2 g (24 mmol) of Fmoc-beta-ala-wang resin was swollen in DMF (480 ml) and after 10 min piperidine (120 ml, 20% solution in DMF) was added. The mixture was shaken for 55 min and drained by suction in a glass filter funnel and washed with DMF (3×300 ml) and NMP (3×300 ml). The reaction was repeated once more.

To the resin-bound intermediate (approximately 40 g, 24 mmol) was added a solution of 4-fluoro-3-nitrobenzoic acid (88.9 g, 480 mmol, 20 eq) in a mixture of 1,2-dichloro-propane and NMP (1:2; 510 ml, dissolved by sonication for 15-20 min). Then DIC (37.4 ml, 240 mmol, 10 eq) was added and the reaction was shaken for two days. The resin was drained and washed with NMP (3×300 ml) and DCM (6×300 ml), and dried for 3 days at 40 ° C. in a vacuum oven to give the resin-bound 3-(4-fluoro-3-nitrobenzoylamino)propionic acid.

1.0 g of this resin was weighed in a Teflon reactor and swelled in NMP (15 ml), drained and re-swelled. To the resin was added 2.19 g 3,5-di(trifluoromethyl)benzylamine (9 mmol, 15 eq) in NMP (9 ml) and the mixture was shaken for 3 days. The resin was drained and washed with NMP (5×12 ml). To the drained resin was added a solution of 3-(3-tri-fluoromethyl)phenoxybenzaldehyde (12 mmol, 20 eq) in NMP (7.5 ml). Then the resin was added a freshly made solution of stannous chloride (1.36 g, 6 mmol, 10 eq) in NMP (6.5 ml) and shaken overnight. The resin was drained and washed with NMP (5×12 ml) and DCM (10×12 ml). The resin was added a 1:1 solution of TFA and DCM (12 ml) and the mixture was shaken for 45 min. The resin was drained and the filtrate and washings (DCM, 3×12 ml) were collected and evaporated. The residue was re-dissolved and evaporated three times more from DCM.

The residue was purified twice by column chromatography using silica gel and a mixture of DCM and MeOH (90:10) as eluent followed by preparative HPLC to afford the title compound (0.024 g).

¹H NMR (DMSO-d₆): δ2.54 (2H, m, below DMSO), 3.50 (2H, m, below water), 5.83 (2H, s), 7.24 (1H, s), 7.30 (3H, m), 7.47 (1H, m), 7.51-7.68 (6H, m), 7.80 (1H, dd), 7.98 (1H, s), 8.27 (1H, s), 8.54 (1H, t), 12.2 (1H, broad); HPLC-MS (Method A): m/z=696 (M+1); Rₜ=6.85 min.

### Example 27

3-{[2-(4-Dibutylaminophenyl)-1-(2-phenoxyethyl)-1H-benzimidazole-5-carbonyl]amino}-propionic acid

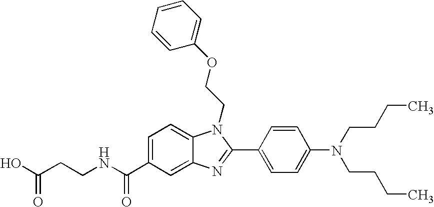

1.0 g of the above resin (example 26) was weighed in a Teflon reactor and swelled in NMP (15 ml), drained and re-swelled. To the resin was added a solution of 2-phenoxy-ethylamine (1.23 g, 9 mmol, 15 eq) in NMP (10 ml) and the mixture was shaken for 3 days. The resin was drained and washed with NMP (5×12 ml). To the resin was added a solution of 4-(dibutylamino)benzaldehyde (2.86 ml, 12 mmol, 20 eq) in NMP (7 ml). Then the resin was added a freshly made solution of stannous chloride (1.36 g, 6 mmol, 10 eq) in NMP (6.5 ml) and the reaction mixture was shaken overnight. The resin was drained and washed with NMP (5×12 ml) and DCM (10×12 ml). The resin was added a 1:1 mixture of TFA and DCM (12 ml) and shaken for 45 min. The resin was drained and the filtrate and washings (DCM, 3×12 ml) were collected and evaporated. The residue was re-dissolved and evaporated three times more with DCM. The residue was purified by column chromatography using 38 g of silica and a mixture of DCM and MeOH (90:10) as eluent to give the title compound (0.12 g).

¹H NMR (DMSO-d₆): δ0.91-0.99 (6H, m), 1.30-1.43 (4H, m), 1.50-1.63(4H, m), 2.53-2.60 (2H, m, below DMSO), 3.35-3.46 (4H, m), 3.47-3.58 (2H, m), 4.41-4.49 (2H, m), 4.80-4.89 (2H, m), 6.78-6.84 (2H, m), 6.85-6.93 (3H, m), 7.18-7.27 (2H, m), 7.77 (2H, d), 7.96-8.10 (2H, dd), 8.17 (1H, s), 8.70 (1H, t); HPLC-MS (Method A): m/z=557(M+1); Rₜ=5.92 min.

The following compounds of the invention were prepared according to general procedure (B) on an Advanced Chem Tech synthesiser and found as hits (defined as compounds that show at least 40% displacement of radiotracer at 1 μM compound concentration) in Glucagon Binding Assay (II)):

### Example 28

3-Benzyloxy-2-({1-[2-(4-bromophenyl)ethyl]-2-[3-(4-methoxyphenoxy)phenyl]-1H-benzimidazole-5-carbonyl}amino)propionic acid

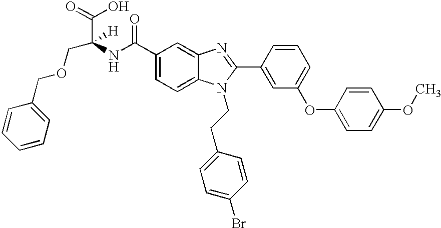

### Example 29

3-Benzyloxy-2-{[1-(2,4-dichloro-6-methylbenzyl)-2-(2,4-difluorophenyl)-1H-benzimidazole-5-carbonyl]amino}propionic acid

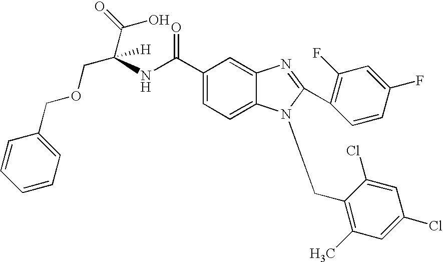

### Example 30

3-Benzyloxy-2-{[2-(5-bromo-2-methoxyphenyl)-1-(2,4-dichloro-6-methylbenzyl)-1H-benzimidazole-5-carbonyl]amino}propionic acid

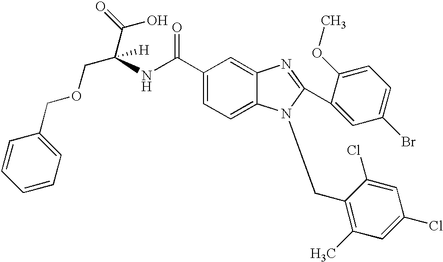

### Example 31

3-Benzyloxy-2-({2-(5-bromo-2-methoxyphenyl)-1-[2-(2-chlorophenoxy)ethyl]-1H-benzimidazole-5-carbonyl}amino)propionic acid

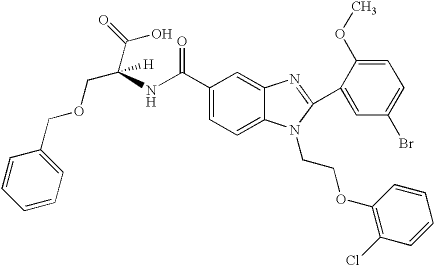

### Example 32

3-{[2-(4-Benzyloxy-3-methoxyphenyl)-1-(4-trifluoromethoxybenzyl)-1H-benzimidazole-5-carbonyl]amino}propionic acid

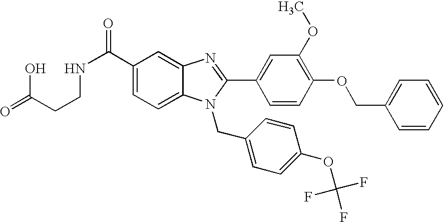

### Example 33

3-{[2-(4-Benzyloxy-3-methoxyphenyl)-1-(3-trifluoromethylbenzyl)-1H-benzimidazole-5-carbonyl]amino}propionic acid

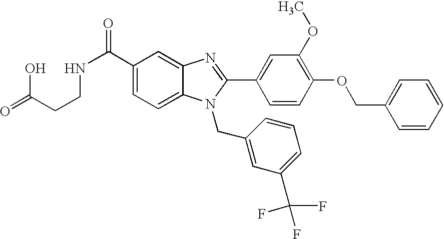

### Example 34

3-{[2-(4-tert-Butylphenyl)-1-(3-phenylpropyl)-1H-benzimidazole-5-carbonyl]amino}propionic acid

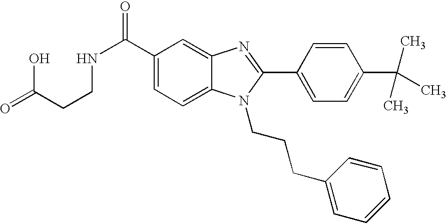

### Example 35

3-{[2-(3-Benzyloxy-4-methoxyphenyl)-1-(3,5-dichlorobenzyl)-1H-benzimidazole-5-carbonyl]amino}propionic acid

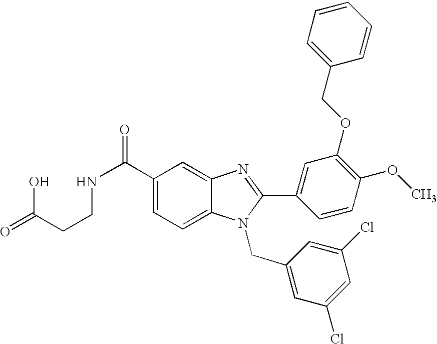

### Example 36

3-({2-(4-tert-Butylphenyl)-1-[2-(2-chlorophenoxy)-ethyl]-1H-benzimidazole-5-carbonyl}amino)propionic acid

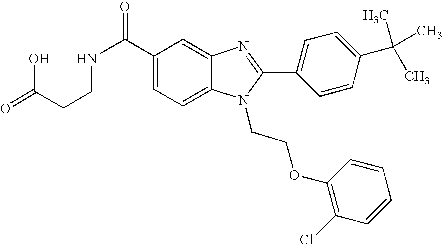

### Example 37

3-Hydroxy-2-{[2-[2-(2-methoxyphenyl)vinyl]-1-(4-phenylbutyl)-1H-benzimidazole-5-carbonyl]amino}propionic acid

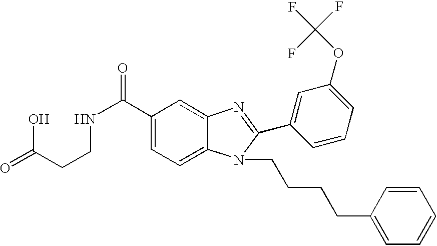

### Example 38

3-{[2-(3,5-Difluorophenyl)-1-(1,2-diphenylethyl)-1H-benzimidazole-5-carbonyl]-amino}propionic acid

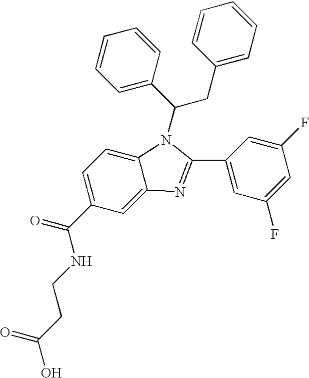

### Example 39

3-({1-(2-Cyclohex-1-enylethyl)-2-[2-(2,6-dichlorobenzyloxy)phenyl]-1H-benzimidazole-5-carbonyl}amino)propionic acid

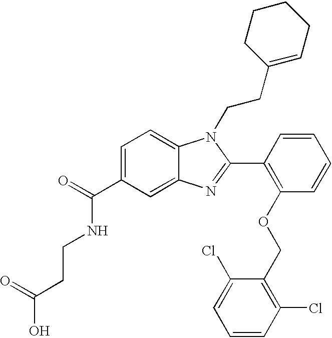

### Example 40

3-{[2-Bicyclo[2.2.1]hept-5-en-2-yl-1-(3,5-bis(trifluoromethyl)benzyl)-1H-benzimidazol-5-carbonyl]amino}propionic acid

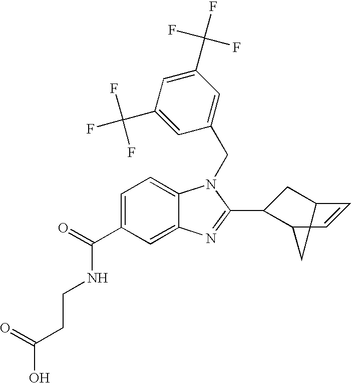

### Example 41

3-({1-(3,5-Bis(trifluoromethyl)benzyl)-2-[5-(3-chlorophenyl)furan-2-yl]-1H-benzimidazole-5-carbonyl}amino)propionic acid

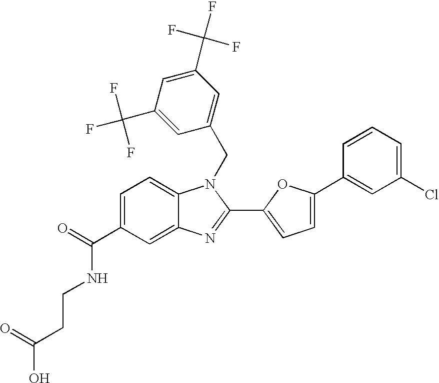

### Example 42

3-({1-(3,5-Bis(trifluoromethyl)benzyl)-2-[5-(2-chlorophenyl)furan-2-yl]-1H-benzimidazole-5-carbonyl}amino)propionic acid

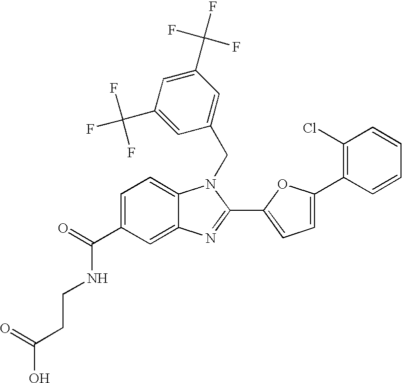

### Example 43

3-{[1-(3,5-Bis(trifluoromethyl)benzyl)-2-(4-dibutylaminophenyl)-1H-benzimidazole-5-carbonyl]amino}propionic acid

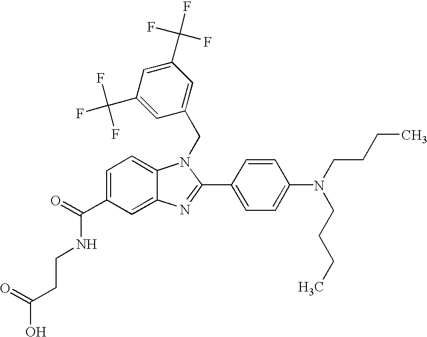

### Example 44

3-{[1-(3,5-Bis(trifluoromethyl)benzyl)-2-(3-trifluoromethylphenyl)-1H-benzimidazole-5-carbonyl]amino}propionic acid

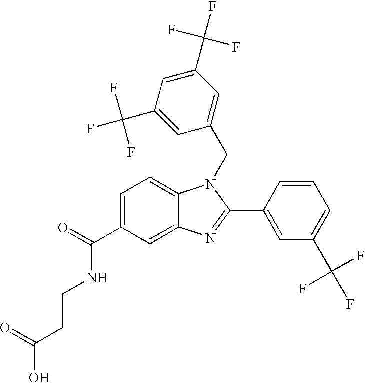

### Example 45

3-({1-(3,5-Bis(trifluoromethyl)benzyl)-2-[3-(3-trifluoromethylphenoxy)phenyl]1H-benzimidazole-5-carbonyl}amino)propionic acid

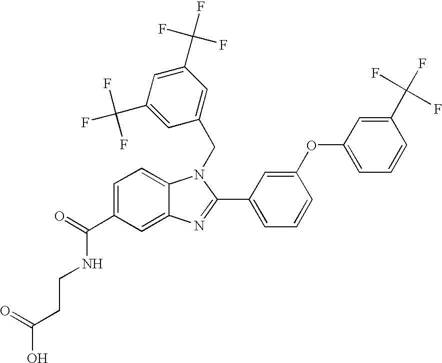

### Example 46

3-({1-(3,5-Bis(trifluoromethyl)benzyl)-2-[4-(2-chloro-6-fluorobenzyloxy)phenyl]-1H-benzimidazole-5-carbonyl}amino)propionic acid

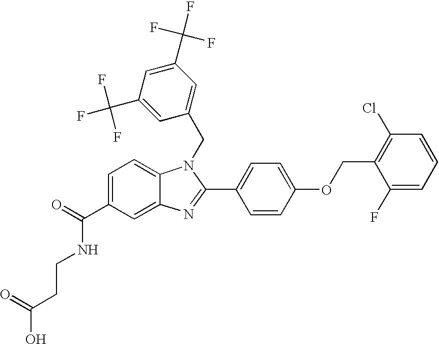

### Example 47

3-{[1-(3,5-Bis(trifluoromethyl)benzyl)-2-(3,5-difluorophenyl)-1H-benzimidazole-5-carbonyl]amino}propionic acid

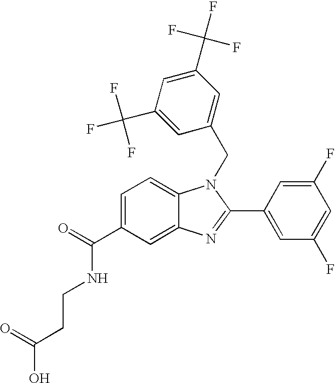

### Example 48

3-{[1-(3,5-Bis(trifluoromethyl)benzyl)-2-(4-chloro-3-nitrophenyl)-1H-benzimidazole-5-carbonyl]amino}propionic acid

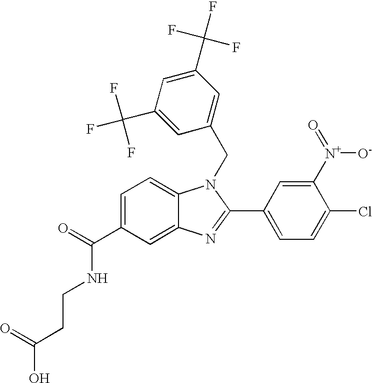

### Example 49

3-{[1-(3,5-Bis(trifluoromethyl)benzyl)-2-(2-chloro-4-dimethylaminophenyl)-1H-benzimidazole -5-carbonyl]amino}propionic acid

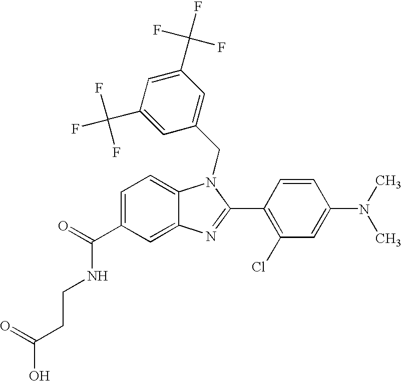

### Example 50

3-{[1-(3,5-Bis(trifluoromethyl)benzyl)-2-(2-butyl-1H-imidazol-4-yl)-1H-benzimidazole -5-carbonyl]amino}propionic acid

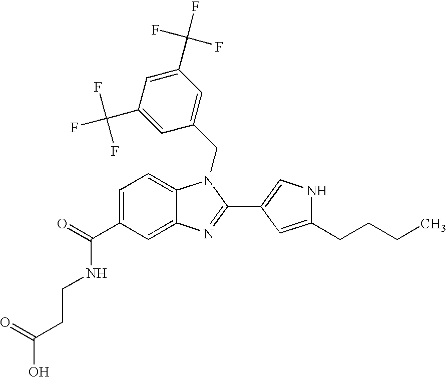

### Example 51

3-{[2-(4-Dibutylaminophenyl)-1-(2-phenoxyethyl)-1H-benzimidazole-5-carbonyl]amino}propionic acid

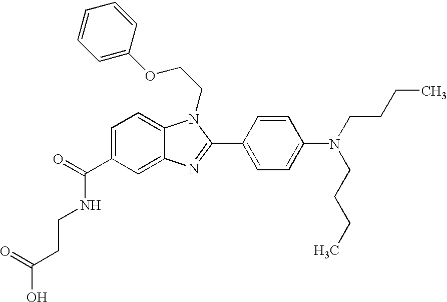

### Example 52

3-({1-(2-Phenoxyethyl)-2-[3-(3-trifluoromethylphenoxy)phenyl]-1H-benzimidazole-5-carbonyl}amino)propionic acid

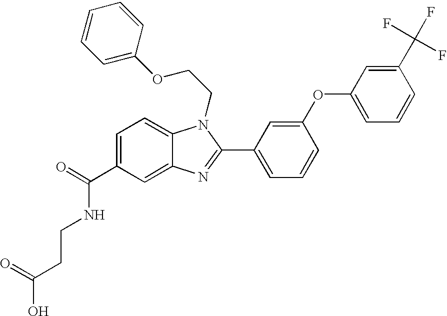

### Example 53

3-{[2-(2-Chloro-4-dimethylaminophenyl)-1-(2-phenoxyethyl)-1H-benzimidazole-5-carbonyl]amino}propionic acid

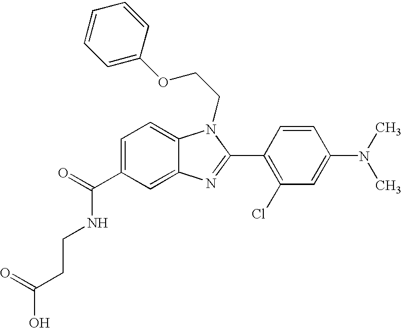

### Example 54

3-{[2-(4-Bromothiophen-2-yl)-1-(4-trifluoromethoxybenzyl)-1H-benzimidazole-5-carbonyl]amino}propionic acid

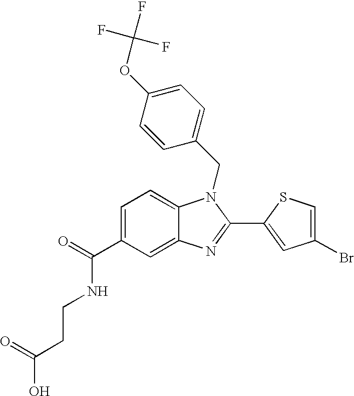

### Example 55

3-{[2-(9H-Fluoren-2-yl)-1-(4-trifluoromethoxybenzyl)-1H-benzimidazole-5-carbonyl]amino}propionic acid

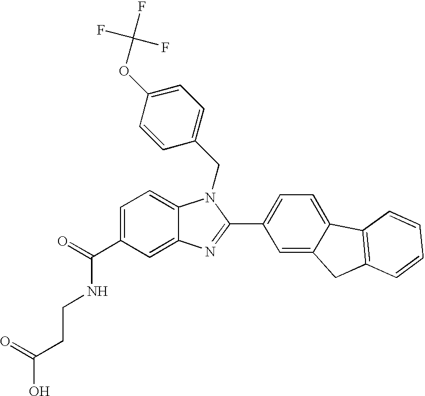

### Example 56

3-{[2-[5-(3-Chlorophenyl)furan-2-yl]-1-(3,4-dichlorobenzyl)-1H-benzimidazole-5-carbonyl]amino}propionic acid

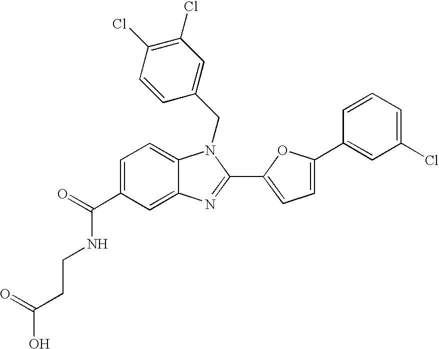

### Example 57

9025-0092-0282 3-{[2-[5-(2-Chlorophenyl)furan-2-yl]-1-(3,4-dichlorobenzyl)-1H-benzimidazole-5-carbonyl]amino}propionic acid

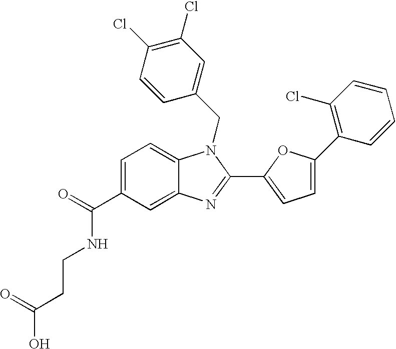

### Example 58

3-{[2-(4-Dibutylaminophenyl)-1-(3,4-dichlorobenzyl)-1H-benzimidazole-5-carbonyl]-amino}propionic acid

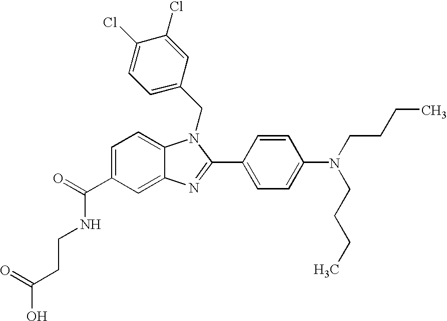

### Example 59

3-({1-(3,4-Dichlorobenzyl)-2-[3-(3-trifluoromethylphenoxy)phenyl]-1H-benzimidazole-5-carbonyl}amino)propionic acid

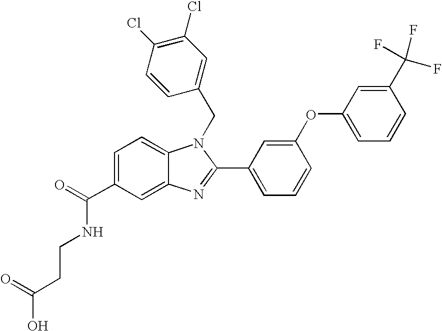

### Example 60

3-{[2-(2-Chloro-4-dimethylaminophenyl)-1-(3,4-dichlorobenzyl)-1H-benzimidazole-5-carbonyl]amino}propionic acid

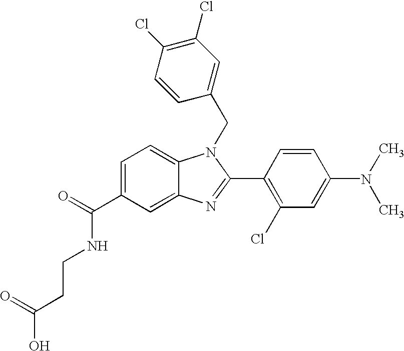

### Example 61

3-({1-[2-(2-Chlorophenoxy)ethyl]-2-[2-(2,6-dichlorobenzyloxy)phenyl]-1H-benzimidazole-5-carbonyl}amino)propionic acid

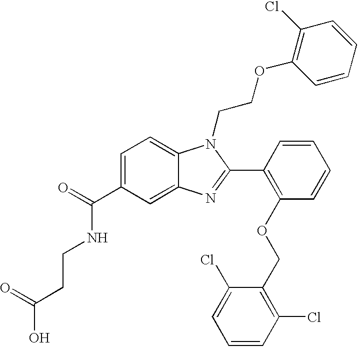

### General Procedure (C)

The compounds of formula (I₂) may be prepared on solid support using the following procedure:

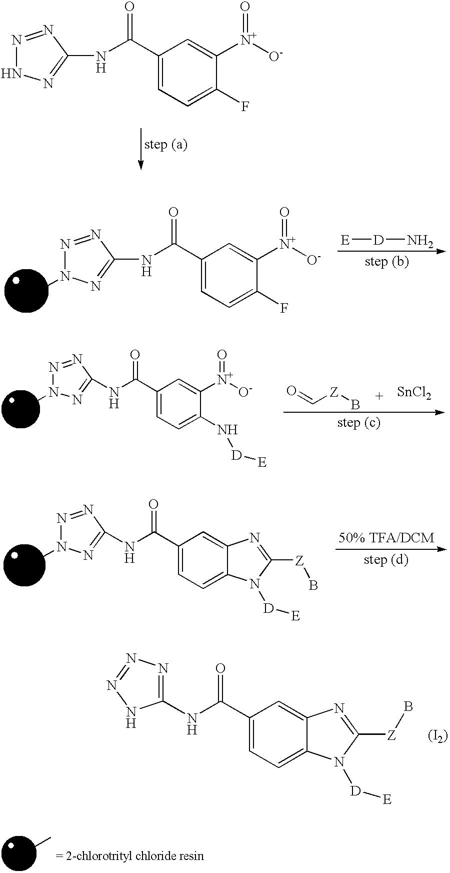

wherein Z, B, D and E are as defined for formula (I).

Attachment of 4-fluoro-3-nitro-N-2H-tetrazol-5-yl)benzamide to a trityl resin is performed in step (a). This can be done by reaction of 4-fluoro-3-nitro-N-2H-tetrazol-5-yl)benzamide with a 2-chlorotrityl chloride resin in eg NMP in the presence of a base, such as DIPEA. In the following step (b) an aromatic nucleophilic substitution with an amine is carried out in an aprotic solvent such as DMSO. The benzimidazole formation in step (c) can be performed as a one-pot reaction by adding an aldehyde together with a reducing reagent (eg stannous chloride dihydrate). Cleavage of the final compound of formula (I₁) in step (d) can be performed under acidic conditions using eg TFA in DCM.

Protocol for Synthesis of Compounds of Formula (I₂) According to General Procedure (C):

Step (a): Attachment of 4-fluoro-3-nitro-N-2H-tetrazol-5-yl)benzamide to Trityl or Chlorortrityl Resin

4-Fluoro-3-nitro-N-2H-tetrazol-5-yl)benzamide in NMP is added to a 2-chlorotrityl chloride resin followed by the addition of a 0.05 M solution of DIPEA in NMP and the mixture is shaken at room temperature for 15 hours. The excess of reagents is removed by filtration and the resin is washed with NMP.

Step (b): Nucleophilic Aromatic Substitution of Aromatic Fluoride with an Amine

1500 μl of a 0.9 M amine solution in DMSO is transferred to the resin. The reaction is run at room temperature for 9.5 hours at 450 rpm. The well is emptied and washed once with NMP (1×1500 μl) and then with DCM (3×1500 μl).

Step (c): Benzimidazole Formation

1 ml of 0.5 M aldehyde solution in NMP is added followed by 2 ml of a fresh 1.1 M solution of stannous chloride dihydrate in NMP. The resulting mixture is shaken at room temperature under exclusion of air for 15 hours. The resin is drained and washed with NMP (4×1 ml).

Step (d): Cleavage

1200 μl TFA solution is added to the resin. The mixture is shaken and left for one hour. The well is emptied for 5 min into a cleavage vial and concentrated in vacuo.

Pharmacological Methods

In the following section binding assays as well as functional assays useful for evaluating the efficiency of the compounds of the invention are described.

Binding of compounds to the glucagon receptor may be determined in a competition binding assay using the cloned human glucagon receptor.

Antagonism may be determined as the ability of the compounds to inhibit the amount of cAMP formed in the presence of 5 nM glucagon.

Glucagon Binding Assay (I)

Receptor binding is assayed using cloned human receptor (Lok et al., Gene 140, 203-209 (1994)). The receptor inserted in the pLJ6′ expression vector using EcoRI/SSt1 restriction sites (Lok et al.) is expressed in a baby hamster kidney cell line (A3 BHK 570-25). Clones are selected in the presence of 0.5 mg/ml G-418 and are shown to be stable for more than 40 passages. The K_{d} is shown to be 0.1 nM.

Plasma membranes are prepared by growing cells to confluence, detaching them from the surface and resuspending the cells in cold buffer (10 mM tris/HCl, pH 7.4 containing 30 mM NaCl, 1 mM dithiothreitol, 5 mg/l leupeptin (Sigma), 5 mg/l pepstatin (Sigma), 100 mg/l bacitracin (Sigma) and 15 mg/l recombinant aprotinin (Novo Nordisk A/S)), homogenization by two 10-s bursts using a Polytron PT 10-35 homogenizer (Kinematica), and centrifugation upon a layer of 41 w/v % sucrose at 95.000×g for 75 min. The white band located between the two layers is diluted in buffer and centrifuged at 40.000×g for 45 min. The precipitate containing the plasma membranes is suspended in buffer and stored at −80° C. until use.

Glucagon is iodinated according to the chloramine T method (Hunter and Greenwood, Nature 194, 495 (1962)) and purified using anion exchange chromatography (Jrgensen et al., Hormone and Metab. Res. 4, 223-224 (1972). The specific activity is 460 μCi/μg on the day of iodination. Tracer is stored at −18° C. in aliquots and used immediately after thawing.

Binding assays are carried out in triplicate in filter microtiter plates (MADV N65, Millipore). The buffer is 50 mM HEPES, 5 mM EGTA, 5 mM MgCl₂, 0.005% tween 20, pH 7.4. Glucagon is dissolved in 0.05 M HCl, added an equal amount (w/w) of human serum albumin and freeze-dried. On the day of use, it is dissolved in water and diluted in buffer to the desired concentrations.

Test compounds are dissolved and diluted in DMSO. 140 μl buffer, 25 μl glucagon or buffer, and 10 μl DMSO or test compound are added to each well. Tracer (50.000 cpm) is diluted in buffer and 25 μl is added to each well. 1-4 μg freshly thawed plasma membrane protein diluted in buffer is then added in aliquots of 25 μl to each well. Plates are incubated at 30° C. for 2 hours. Non-specific binding is determined with 10⁻⁶ M of glucagon. Bound tracer and unbound tracer are then separated by vacuum filtration (Millipore vacuum manifold). The plates are washed with 2×100 μl buffer/well. The plates are air dried for a couple of hours, whereupon the filters are separated from the plates using a Millipore Puncher. The filters are counted in a gamma counter.

Functional Assay (I)

The functional assay is carried out in 96 well microtiter plates (tissue culture plates, Nunc). The resulting buffer concentrations in the assay are 50 mM tris/HCl, 1 mM EGTA, 1.5 mM MgSO₄, 1.7 mM ATP, 20 μM GTP, 2 mM IBMX, 0.02% tween-20 and 0.1% human serum albumin. pH was 7.4. Glucagon and proposed antagonist are added in aliquots of 35 μl diluted in 50 mM tris/HCl, 1 mM EGTA, 1.85 mM MgSO₄, 0.0222% tween-20 and 0.111% human serum albumin, pH 7.4. 20 μl of 50 mM tris/HCl, 1 mM EGTA, 1.5 mM MgSO₄, 11.8 mM ATP, 0.14 mM GTP, 14 mM IBMX and 0.1% human serum albumin, pH 7.4 was added. GTP was dissolved immediately before the assay.

50 μl containing 5 μg of plasma membrane protein was added in a tris/HCl, EGTA, MgSO₄, human serum albumin buffer (the actual concentrations are dependent upon the concentration of protein in the stored plasma membranes).

The total assay volume is 140 μl. The plates are incubated for 2 hours at 37° C. with continuous shaking. Reaction is terminated by addition of 25 μl 0.5 N HCl. cAMP is measured by the use of a scintillation proximity kit (Amersham).

Glucagon Binding Assay (II)

BHK (baby hamster kidney cell line) cells are transfected with the human glucagon receptor and a membrane preparation of the cells is prepared. Wheat Germ Agglutinin derivatized SPA beads containing a scintillant (WGA beads) (Amersham) bound the membranes. ¹²⁵I-glucagon bound to human glucagon receptor in the membranes and excited the scintillant in the WGA beads to light emission. Glucagon or samples binding to the receptor competed with ¹²⁵I-glucagon.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000×g) the supernatant is transferred to cold centrifuge tubes and centrifuged for 45 min at 40.000×g. The pellet is resuspended in homogenisation buffer, homogenised 2×10 sec (Polytron) and additional homogenisation buffer is added. The suspension is centrifuged for 45 min at 40.000×g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/ml. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The glucagon binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl glucagon or test compound (in DMSO) are added to each well. 50 μl tracer (¹²⁵I-porcine glucagon, 50.000 cpm) and 50 μl membranes (7.5 μg) containing the human glucagon receptor are then added to the wells. Finally 50 μl WGA beads containing 1 mg beads are transferred to the well. The opti plates are incubated for 4 hours on a shaker and then settled for 8-48 hours. The opti plates are counted in a Topcounter. Non-specific binding is determined with 500 nM of glucagon

Many of the compounds according to the examples showed IC₅₀ values below 1000 nM when tested in the glucagon binding assay (II).

GIP Binding Assay

BHK (baby hamster kidney cell line) cells are transfected with the human GIP receptor and a membrane preparation of the cells is prepared. Wheat Germ Agglutinin derivatized SPA beads containing a scintillant (WGA beads) (Amersham) bound the membranes. ¹²⁵I-GIP bound to human GIP receptor in the membranes and excited the scintillant in the WGA beads to light emission. GIP or samples binding to the receptor competed with ¹²⁵I-GIP.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000×g) the supernatant is transferred to cold centrifuge tubes and centrifuged for 45 min at 40.000×g. The pellet is resuspended in homogenisation buffer, homogenised 2×10 sec (Polytron) and additional homogenisation buffer is added. The suspension is centrifuged for 45 min at 40.000×g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/ml. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The GIP binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl GIP or test compound (in DMSO) are added to each well. 50 μl tracer (¹²⁵I-porcine GIP, 50.000 cpm) and 50 μl membranes (20 μg) containing the human GIP receptor are then added to the wells. Finally 50 μl WGA beads containing 1 mg beads are transferred to the well. The opti plates are incubated for 3.5 hours on a shaker and then settled for 8-48 hours. The opti plates are counted in a Topcounter. Non-specific binding is determined with 500 nM of GIP.

Generally, the compounds show a higher affinity for the glucagon receptor compared to the GIP receptor.

1. A compound of formula (I): 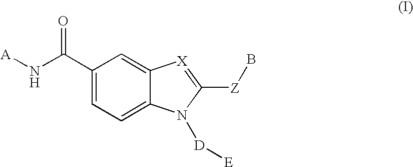
wherein
A is 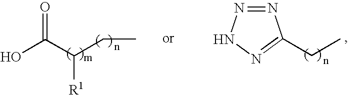
m is 0 or 1,
n is 0, 1, 2 or 3,
with the proviso that m and n must not both be 0,
R¹ is hydrogen, fluoro or —(CH₂)ₒ—OR²,
o is 0 or 1,
R² is hydrogen, C₁₋₆-alkyl, C₁₋₆-alkanoyl, aryl or aryl-C₁₋₆-alkyl,
X is —N═ or —CH═,
B is 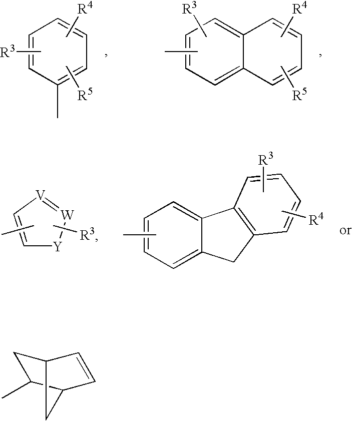
V and W independently are —CH═ or —N═,
Y is —O—, —S— or —NH—,
R³, R⁴ and R⁵ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂R⁷, —S(O)₂NR⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,
C₁₋₆-alkyl, C₂₋₆-alkenyl or C₂₋₆-alkynyl,
which may optionally be substituted with one or more substituents selected from fluoro, —CN, —CF₃, —OCF₃, —OR⁶ and —NR⁶R⁷,
C₃₋₈-cycloalkyl, C₄₋₈-cycloalkenyl, heterocyclyl, C₃₋₈-cycloalkyl-C₁₋₆-alkyl, C₃₋₈-cyloalkyl -C₁₋₆-alkoxy, C₃₋₈-cycloalkyloxy, C₃₋₈-cycloalkyl-C₁₋₆-alkylthio, C₃₋₈-cycloalklthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,
aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R³ to R⁵ when placed in adjacent positions together may form a bridge —(CR⁸R⁹)ₛ—O—(CR¹⁰R¹¹)ₜ—O—,
s is 0, 1 or 2,
t is 1 or 2,
R⁸, R⁹, R¹⁰ and R¹¹ independently are hydrogen, C₁₋₆-alkyl or fluoro,
D is —(CH₂)ₚ—, 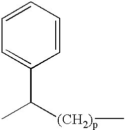
or —(CH₂)ₚ—O—,
p is 0, 1, 2, 3 or 4,
E is 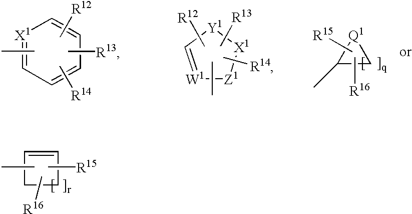
X¹, Z¹ and W¹ independently are —CH═ or —N═,
Y¹ is —O—, —S— or —NH—,
Q¹ is —CH₂— or —NH—,
q is 2, 3, 4, 5 or 6,
r is 1, 2, 3, 4 or 5,
R¹², R¹³ and R¹⁴ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —C(O)R¹⁷ or —C(O)OR¹⁷ ,
C₁₋₆-alkyl, C₂₋₆-alkenyl or C₂₋₆-alkynyl,
which may optionally be substituted with one or more substituents selected from fluoro, —CN, —CF₃, —OCF₃, —OR¹⁷ and —NR¹⁷R¹⁸,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR¹⁷, —CN, —CF₃, —OCF₃, —OR¹⁷ and —NR¹⁷R¹⁸,
aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,
R¹⁷ and R¹⁸ independently are hydrogen or C₁₋₆-alkyl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹² to R¹⁴ when placed in adjacent positions together may form a bridge —(CR¹⁹R²⁰)ₓ—O—(CR²¹R²²)_{y}—O—,
x is 0, 1 or 2,
y is 1 or 2,
R¹⁹, R²⁰, R²¹ and R²² independently are hydrogen, C₁₋₆-alkyl or fluoro,
R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,
wherein the cyclic moieties may optionally be substituted with one or more substituents selected from halogen, —CN, —CF₃, —NO₂, —OR²³, —NR²³R²⁴ and C₁₋₆-alkyl,
R²³ and R²⁴ independently are hydrogen or C₁₋₆-alkyl, or
R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or E is
C₁₋₆-alkyl, C₂₋₆-alkenyl or C₂₋₆-alkynyl,
which may optionally be substituted with one or more substituents selected from halogen, —CN, —CF₃, —OCF₃, —NO₂, —OR²⁵,—SR²⁵, —NR²⁵R²⁶ and C₁₋₆-alkyl,
R²⁵ and R²⁶ independently are hydrogen or C₁₋₆-alkyl, or
R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
Z is —(CR²⁷R²⁸)ᵥ—(O)_{w}—(CR²⁹R³⁰)_{z}—,
v and z independently are 0, 1 or 2,
w is 0 or 1,
R²⁷, R²⁸, R²⁹ and R³⁰ independently are hydrogen or C₁₋₆-alkyl,
with the proviso that the compound must not be 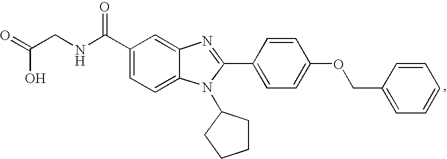
as well as any diastereomer or enantiomer or tautomeric form thereof including mixtures of these or a pharmaceutically acceptable salt thereof.

2. A compound according to claim 1, wherein A is

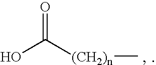

3. A compound according to claim 2, wherein A is

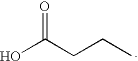

4. A compound according to claim 1, wherein A is

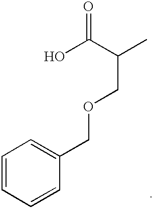

5. A compound according to claim 1, wherein B is

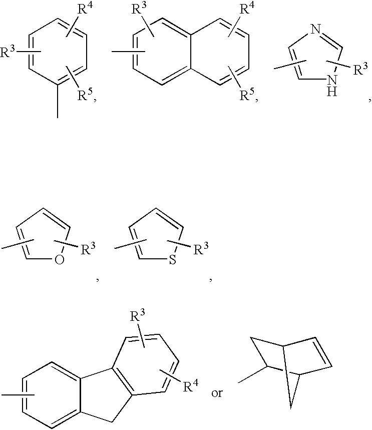

6. A compound according to claim 5, wherein B is 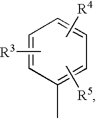
wherein R³, R⁴ and R⁵ independently are
hydrogen, halogen, —CF₃, —OCF₃, —NO₂, —OR⁶, —NR⁶R⁷, C₁₋₆-alkyl,
aryloxy, aryl-C₁₋₆-alkoxy,
of which the cyclic moieties may optionally be substituted with one or more substituents selected from halogen, —CF₃, —OCF₃, —NO₂, —OR⁶, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

7. A compound according to claim 6, wherein B is 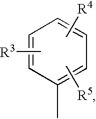
wherein
R³, R⁴ and R⁵ independently are
hydrogen, halogen, —CF₃, —OCF₃, —NO₂, —OR⁶, —NR⁶R⁷, C₁₋₆-alkyl,
phenoxy, phenyl-C₁₋₆-alkoxy,
of which the cyclic moieties optionally may be substituted with one or more substituents selected from halogen, —CF₃, —OCF₃, —NO₂, —OR⁶, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

8. A compound according to claim 7, wherein B is 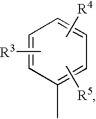
wherein
R³, R⁴ and R⁵ independently are
hydrogen, halogen, —CF₃, —OCF₃, —NO₂, —OR⁶, —NR⁶R⁷, C₁₋₆-alkyl,
phenoxy, phenyl-C₁₋₆-alkoxy,
of which the cyclic moieties may optionally be substituted with one or more substituents selected from halogen, —CF₃, and C₁₋₆-alkoxy,
R⁶ and R⁷ independently are hydrogen or C₁₋₆-alkyl.

9. A compound according to claim 6, wherein R³ is hydrogen, and R⁴ and R⁵ are different from hydrogen.

10. A compound according to claim 6, wherein R³ and R⁴ are hydrogen, and R⁵ is different from hydrogen.

11. A compound according to claim 5, wherein B is 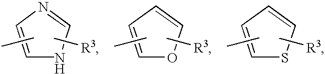
wherein R³ is
hydrogen, halogen, C₁₋₆-alkyl,
aryl, which may optionally be substituted with one or more substituents selected from halogen, —CF₃, —OCF₃, —NO₂, —OR⁶, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

12. A compound according to claim 11, wherein B is 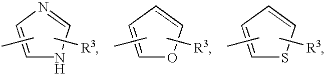
wherein R³ is
hydrogen, halogen, C₁₋₆-alkyl,
phenyl, which may optionally be substituted with one or more substituents selected from halogen, —CF₃, —OCF₃, —NO₂, —OR⁶, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds.

13. A compound according to claim 12, wherein B is 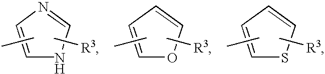
wherein R³ is
hydrogen, halogen, C₁₋₆-alkyl,
phenyl, which is substituted with one halogen substituent.

14. A compound according to claim 5, wherein B is

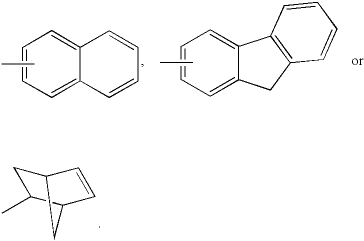

15. A compound according to claim 1, wherein Z is a valence bond, —CH₂—, —(CH₂)₂—, —(CH₂)₃—, —CH(CH₃)— or —CH(CH₃)—O—.

16. A compound according to claim 15, wherein Z is a valence bond.

17. A compound according to claim 1, wherein D is a valence bond, —CH₂—, —(CH₂)₂—, —(CH₂)₃—, —(CH₂)₄—, —(CH₂) ₂—O— or

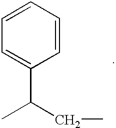

18. A compound according to claim 17, wherein D is a valence bond, —CH₂— or —(CH₂)₂—O—.

19. A compound according to claim 18, wherein D is —CH₂— or —(CH₂)₂—O—.

20. A compound according to claim 1, wherein E is

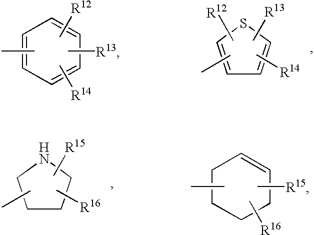

21. A compound according to claim 20, wherein E is 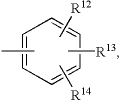
wherein R¹², R¹³ and R¹⁴ independently are hydrogen, halogen, —CF₃, —OCF₃, C₁₋₆-alkyl, C₃₋₈-cycloalkyl, C₄₋₈-cycloalkenyl or aryl.

22. A compound according to claim 21, wherein E is 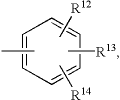
wherein R¹², R¹³ and R¹⁴ independently are hydrogen, halogen, —CF₃, —OCF₃ or C₁₋₆-alkyl.

23. A compound according to claim 22, wherein E is 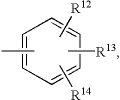
wherein R¹² is hydrogen, and R¹³ and R¹⁴ independently are halogen, —CF₃, —OCF₃ or C₁₋₆-alkyl.

24. A compound according to claim 23, wherein R¹² is hydrogen, and R¹³ and R¹⁴ are both halogen or are both —CF₃.

25. A compound according to claim 22, wherein E is 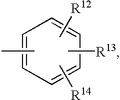
wherein R¹² and R¹³ are both hydrogen, and R¹⁴ is halogen, —CF₃, —OCF₃ or C₁₋₆-alkyl.

26. A compound according to claim 20, wherein E is

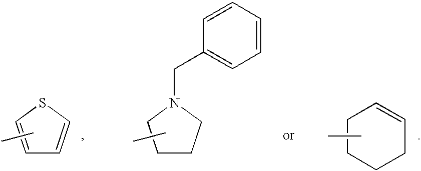

27. A compound according to claim 1, wherein X is —N═.

28. A compound according to claim 1, wherein said compound has an IC₅₀ value of no greater than 5 μM as determined by Glucagon Binding Assay (I) or Glucagon Binding Assay (II).

29. A compound according to claim 28, wherein said compound has an IC₅₀ value of less than 1 μM as determined by Glucagon Binding Assay (I) or Glucagon Binding Assay (II).

30. A compound according to claim 1, which is an agent useful for the treatment of an indication selected from the group consisting of hyperglycemia, impaired glucose tolerance (IGT), type 2 diabetes, type 1 diabetes, dyslipidemia and obesity.

31. A compound according to claim 1 for use as a medicament.

32. A pharmaceutical composition comprising at least one compound according to claim 1 together with one or more pharmaceutically acceptable carriers or excipients.

33. A pharmaceutical composition according to claim 32 in unit dosage form, said composition comprising from about 0.05 mg to about 1000 mg of said compound.

34. Use of a compound of the general formula (I′): 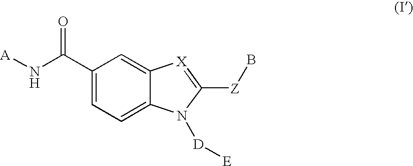
wherein
A is 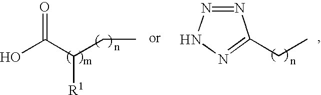
m is 0 or 1,
n is 0, 1, 2 or 3,
with the proviso that m and n must not both be 0,
R¹ is hydrogen, fluoro or —(CH₂)ₒ—OR²,
o is 0 or 1,
R² is hydrogen, C₁₋₆-alkyl, C₁₋₆-alkanoyl, aryl or aryl-C₁₋₆-alkyl,
X is —N═ or —CH═,
B is 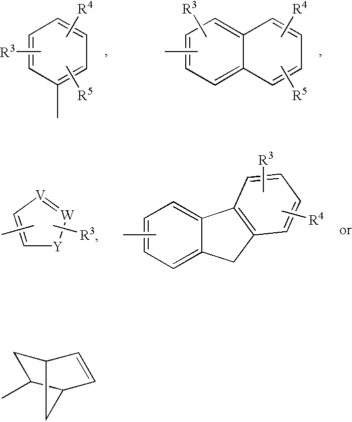
V and W independently are —CH═ or —N═,
Y is —O—, —S— or —NH—,
R³, R⁴ and R⁵ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂NR⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,
C₁₋₆-alkyl, C₂₋₆-alkenyl or C₂₋₆-alkynyl,
which may optionally be substituted with one or more substituents selected from fluoro, —CN, —CF₃, —OCF₃, —OR⁶ and —NR⁶R⁷,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,
aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R³ to R⁵ when placed in adjacent positions together may form a bridge —(CR⁸R⁹)ₛ—O—(CR¹⁰R¹¹)ₜ—O—,
s is 0, 1 or 2,
t is 1 or 2,
R⁸, R⁹, R¹⁰ and R¹¹ independently are hydrogen, C₁₋₆-alkyl or fluoro,
D is —(CH₂)ₚ—, 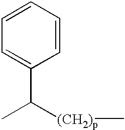
or —(CH₂)ₚ—O—,
p is 0, 1, 2, 3 or 4,
E is 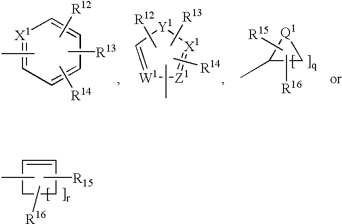
X¹, Z¹ and W¹ independently are —CH═ or —N═,
Y¹ is —O—, —S— or —NH—,
Q¹ is —CH₂— or —NH—,
q is 2, 3, 4, 5 or 6,
r is 1, 2, 3, 4 or 5,
R¹², R¹³ and R¹⁴ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R^{18,}—S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —C(O)R¹⁷ or —C(O)OR¹⁷,
C₁₋₆-alkyl, C₂₋₆-alkenyl or C₂₋₆-alkynyl,
which may optionally be substituted with one or more substituents selected from fluoro, —CN, —CF₃, —OCF₃, —OR¹⁷ and —NR¹⁷R¹⁸,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio; C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR¹⁷, —CN, —CF₃, —OCF₃, —OR¹⁷ and —NR¹⁷R¹⁸,
aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,
R¹⁷ and R¹⁸ independently are hydrogen or C₁₋₆alkyl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹² to R¹⁴ when placed in adjacent positions together may form a bridge —(CR¹⁹R²⁰)ₓ—O—(CR²¹R²²)_{y}—O—,
x is 0, 1 or 2,
y is 1 or 2,
R¹⁹, R²⁰, R²¹ and R²² independently are hydrogen, C₁₋₆-alkyl or fluoro,
R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,
wherein the cyclic moieties may optionally be substituted with one or more substituents selected from halogen, —CN, —CF₃, —NO₂, —OR²³, —NR²³R²⁴ and C₁₋₆-alkyl,
R²³ and R²⁴ independently are hydrogen or C₁₋₆-alkyl, or
R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or E is
C₁₋₆-alkyl, C₂₋₆-alkenyl or C₂₋₆-alkynyl,
which may optionally be substituted with one or more substituents selected from halogen, —CN, —CF₃, —OCF₃, —NO₂, —OR²⁵, —SR²⁵, —NR²⁵R²⁶ and C₁₋₆-alkyl,
R²⁵ and R²⁶ independently are hydrogen or C₁₋₆-alkyl, or
R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
Z is —(CR²⁷R²⁸ )ᵥ—(O)_{w}—(CR²⁹R³⁰)_{z}—,
v and z independently are 0, 1 or 2,
w is 0 or 1,
R²⁷, R²⁸, R²⁹ and R³⁰ independently are hydrogen or C₁₋₆-alkyl,
as well as any diastereomer or enantiomer or tautomeric form thereof including mixtures of these or a pharmaceutically acceptable salt thereof for the preparation of a medicament for the treatment of disorders or diseases, wherein a glucagon antagonistic action is beneficial.

35. Use of a compound as defined in claim 34 for the preparation of a medicament for the treatment of glucagon-mediated disorders and diseases.

36. Use of a compound as defined in claim 34 for the preparation of a medicament for the treatment of hyperglycemia.

37. Use of a compound as defined in claim 34 for the preparation of a medicament for lowering blood glucose in a mammal.

38. Use of a compound as defined in claim 34 for the preparation of a medicament for the treatment of IGT.

39. Use of a compound as defined in claim 34 for the preparation of a medicament for the treatment of type 2 diabetes.

40. Use according to claim 39 for the preparation of a medicament for the delaying or prevention of the progression from IGT to type 2 diabetes.

41. Use according to claim 39 for the preparation of a medicament for the delaying or prevention of the progression from non-insulin requiring type 2 diabetes to insulin requiring type 2 diabetes.

42. Use of a compound as defined in claim 34 for the preparation of a medicament for the treatment of type 1 diabetes.

43. Use of a compound as defined in claim 34 for the preparation of a medicament for the treatment of obesity.

44. Use of a compound as defined in claim 34 for the preparation of a medicament for the treatment of dyslipidemia.

45. Use according to any one of the claims 34 to 44 in a regimen which comprises treatment with a further antidiabetic agent.

46. Use according to any one of the claims 34 to 45 in a regimen which comprises treatment with a further antiobesity agent.

47. Use according to any one of the claims 34 to 46 in a regimen which additionally comprises treatment with a further antihyperlipidemic agent.

48. Use according to any one of the claims 34 to 47 in a regimen which additionally comprises treatment with an antihypertensive agent.

49. A method for treating disorders or diseases wherein a glucagon antagonistic action is beneficial, said method comprising administering to a subject in need thereof an effective amount of a compound of formula (I′): 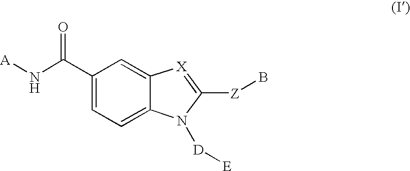
wherein
A is 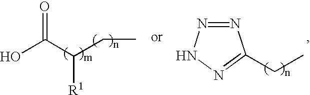
m is 0 or 1,
n is 0, 1, 2 or 3,
with the proviso that m and n must not both be 0,
R¹ is hydrogen, fluoro or —(CH₂)ₒ—OR²,
o is 0 or 1,
R² is hydrogen, C₁₋₆-alkyl, C₁₋₆-alkanoyl, aryl or aryl-C₁₋₆-alkyl,
X is —N═ or —CH═,
B is 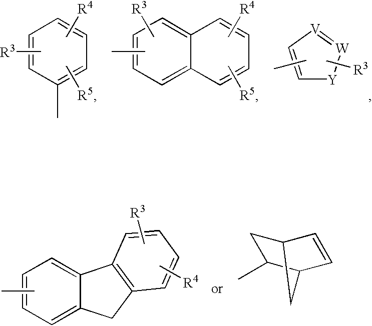
V and W independently are —CH═ or —N═,
Y is —O—, —S— or —NH—,
R³, R⁴ and R⁵ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂R⁷, —S(O)₂NR⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,
C₁₋₆-alkyl, C₂₋₆-alkenyl or C₂₋₆-alkynyl,
which may optionally be substituted with one or more substituents selected from fluoro, —CN, —CF₃, —OCF₃, —OR⁶ and —NR⁶R⁷,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,
aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R³ to R⁵ when placed in adjacent positions together may form a bridge —(CR⁸R⁹)ₛ—O—(CR¹⁰R¹¹)ₜ—O—,
s is 0, 1 or 2,
t is 1 or 2,
R⁸, R⁹, R¹⁰ and R¹¹ independently are hydrogen, C₁₋₆-alkyl or fluoro,
D is —(CH₂)ₚ—, 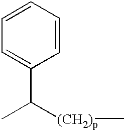
or —(CH₂)ₚ—O—,
p is 0, 1, 2, 3 or 4,
E is 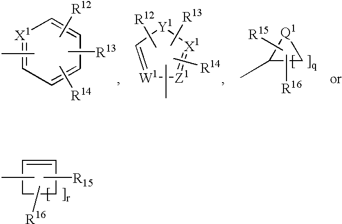
X¹, Z¹ and W¹ independently are —CH═ or —N═,
Y¹ is —O—, —S— or —NH—,
Q¹ is —CH₂— or —NH—,
q is 2, 3, 4, 5 or 6,
r is 1, 2, 3, 4 or 5,
R¹², R¹³ and R¹⁴ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷—S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —C(O)R¹⁷ or —C(O)OR¹⁷,
C₁₋₆-alkyl, C₂₋₆-alkenyl or C₂₋₆-alkynyl,
which may optionally be substituted with one or more substituents selected from fluoro, —CN, —CF₃, —OCF₃, —OR¹⁷ and —NR¹⁷R¹⁸ ,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR¹⁷, —CN, —CF₃, —OCF₃, —OR¹⁷ and —NR¹⁷R¹⁸,
aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,
R¹⁷ and R¹⁸ independently are hydrogen or C₁₋₆-alkyl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹² to R¹⁴ when placed in adjacent positions together may form a bridge —(CR¹⁹R²⁰)ₓ—O—(CR²¹R²²)_{y}—O—,
x is 0, 1 or 2,
y is 1 or 2,
R¹⁹, R²⁰, R²¹ and R²² independently are hydrogen, C₁₋₆-alkyl or fluoro,
R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,
wherein the cyclic moieties may optionally be substituted with one or more substituents selected from halogen, —CN, —CF₃, —NO₂, —OR²³, —NR²³R²⁴ and C₁₋₆-alkyl,
R²³ and R²⁴ independently are hydrogen or C₁₋₆-alkyl, or
R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or E is
C₁₋₆-alkyl, C₂₋₆-alkenyl or C₂₋₆-alkynyl,
which may optionally be substituted with one or more substituents selected from halogen, —CN, —CF₃, —OCF₃, —NO₂, —OR²⁵,—SR²⁵, —NR²⁵R²⁶ and C₁₋₆-alkyl,
R²⁵ and R²⁶ independently are hydrogen or C₁₋₆-alkyl, or
R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
Z is —(CR²⁷R²⁸)ᵥ—(O)_{w}—(CR²⁹R²⁹R³⁰)_{z}—,
v and z independently are 0, 1 or 2,
w is 0 or 1,
R²⁷, R²⁸, R²⁹ and R³⁰ independently are hydrogen or C₁₋₆-alkyl,
as well as any diastereomer or enantiomer or tautomeric form thereof including mixtures of these or a pharmaceutically acceptable salt thereof.

50. The method according to claim 49, wherein the effective amount of the compound is in the range of from about 0.05 mg to about 2000 mg per day.

51. A compound according to claim 28, which has an IC₅₀ value of less than 500 nM as determined by Glucagon Binding Assay (I) or Glucagon Binding Assay (II).

52. A compound according to claim 28, which has an IC₅₀ value of less than 100 nM as determined by Glucagon Binding Assay (I) or Glucagon Binding Assay (II).

53. A pharmaceutical composition according to claim 32 in unit dosage form, said composition comprising from about 0.1 mg to about 500 mg of said compound.

54. A pharmaceutical composition according to claim 32 in unit dosage form, said composition comprising from about 0.5 mg to about 200 mg of said compound.

55. The method according to claim 49, wherein the effective amount of the compound is in the range of from about 0.1 mg to about 1000 mg per day.

56. The method according to claim 49, wherein the effective amount of the compound is in the range of from about 0.5 mg to about 500 mg per day.

57. A method for lowering blood glucose in a mammal, said method comprising administering to said mammal an effective amount of a compound of formula (I′): 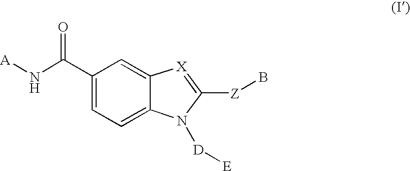
wherein
A is 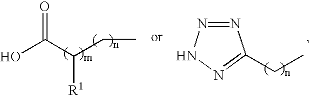
m is 0 or 1,
n is 0, 1, 2 or 3,
with the proviso that m and n must not both be 0,
R¹ is hydrogen, fluoro or —(CH₂)ₒ—OR²,
o is 0 or 1,
R² is hydrogen, C₁₋₆-alkyl, C₁₋₆-alkanoyl, aryl or aryl-C₁₋₆-alkyl,
X is —N═ or —CH═,
B is 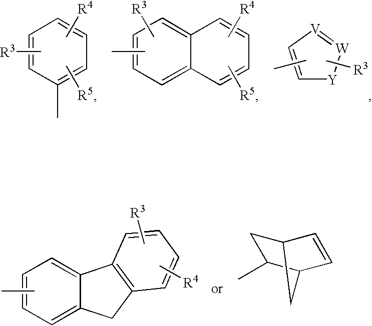
V and W independently are —CH═ or —N═,
Y is —O—, —S— or —NH—,
R³, R⁴ and R⁵ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂R⁷, —S(O)₂NR⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,
C₁₋₆-alkyl, C₂₋₆-alkenyl or C₂₋₆-alkynyl,
which may optionally be substituted with one or more substituents selected from fluoro, —CN, —CF₃, —OCF₃, —OR⁶ and —NR⁶R⁷,
C₃₋₈-cycloalkyl, C₄₋₈-cycloalkenyl, heterocyclyl, C₃₋₈-cycloalkyl-C₁₋₆-alkyl, C₃₋₈-cycloalkyl -C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,
aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R³ to R⁵ when placed in adjacent positions together may form a bridge —(CR⁸R⁹)ₛ—O—(CR¹⁰R¹¹)ₜ—O—,
s is 0, 1 or 2,
t is 1 or 2,
R⁸, R⁹, R¹⁰ and R¹¹ independently are hydrogen, C₁₋₆-alkyl or fluoro,
D is —(CH₂)ₚ—, 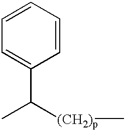
or —(CH₂)ₚ—O—,
p is 0, 1, 2, 3 or 4,
E is 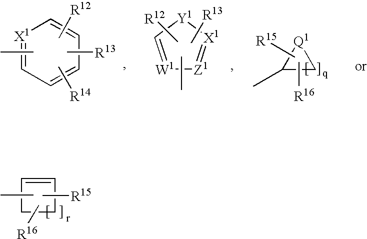
X¹, Z¹ and W¹ independently are —CH═ or —N═,
Y¹ is —O—, —S— or —NH—,
Q¹ is —CH₂— or —NH—,
q is 2, 3, 4, 5 or 6,
r is 1, 2, 3, 4 or 5,
R¹², R¹³ and R¹⁴ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —C(O)R¹⁷ or —C(O)OR¹⁷,
C₁₋₆-alkyl, C₂₋₆-alkenyl or C₂₋₆-alkynyl,
which may optionally be substituted with one or more substituents selected from fluoro, —CN, —CF₃, —OCF₃, —OR¹⁷ and —NR¹⁷R¹⁸,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR¹⁷, —CN, —CF₃, —OCF₃, —OR¹⁷ and —NR¹⁷R¹⁸,
aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,
R¹⁷ and R¹⁸ independently are hydrogen or C₁₋₆-alkyl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹² to R¹⁴ when placed in adjacent positions together may form a bridge —(CR¹⁹R²⁰)ₓ—O—(CR²¹R²²)_{y}—O—,
x is 0, 1 or 2,
y is 1 or 2,
R¹⁹, R²⁰, R²¹ and R²² independently are hydrogen, C₁₋₆-alkyl or fluoro,
R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,
wherein the cyclic moieties may optionally be substituted with one or more substituents selected from halogen, —CN, —CF₃, —NO₂, —OR²³, —NR²³R²⁴ and C₁₋₆-alkyl,
R²³ and R²⁴ independently are hydrogen or C₁₋₆-alkyl, or
R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or E is
C₁₋₆-alkyl, C₂₋₆-alkenyl or C₂₋₆-alkynyl,
which may optionally be substituted with one or more substituents selected from halogen, —CN, —CF₃, —OCF₃, —NO₂, —OR²⁵,—SR²⁵, —NR²⁵R²⁶ and C₁₋₆-alkyl,
R²⁵ and R²⁶ independently are hydrogen or C₁₋₆-alkyl, or
R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
Z is —(CR²⁷R²⁸)ᵥ—(O)_{w}—(CR²⁹R³⁰)_{z}—,
v and z independently are 0, 1 or 2,
w is 0 or 1,
R²⁷, R²⁸, R²⁹ and R³⁰ independently are hydrogen or C₁₋₆-alkyl,
as well as any diastereomer or enantiomer or tautomeric form thereof including mixtures of these or a pharmaceutically acceptable salt thereof.

58. A method for delaying or preventing progression from impaired glucose tolerance to type 2 diabetes, said method comprising administering to a subject in need thereof an effective amount of a compound of formula (I′): 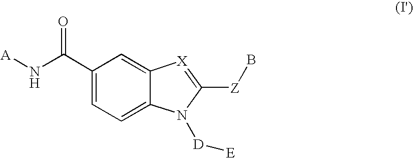
wherein
A is 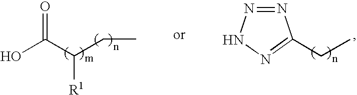
m is 0 or 1,
n is 0, 1, 2 or 3,
with the proviso that m and n must not both be 0,
R¹ is hydrogen, fluoro or —(CH₂)ₒ—OR²,
o is 0 or 1,
R² is hydrogen, C₁₋₆-alkyl, C₁₋₆-alkanoyl, aryl or aryl-C₁₋₆-alkyl,
X is —N═ or —CH═,
B is 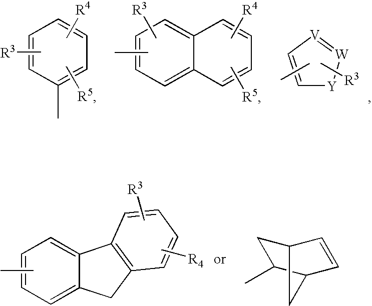
V and W independently are —CH═ or —N═,
Y is —O—, —S— or —NH—,
R³, R⁴ and R⁵ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂R⁷, —S(O)₂NR⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,
C₁₋₆-alkyl, C₂₋₆-alkenyl or C₂₋₆-alkynyl,
which may optionally be substituted with one or more substituents selected from fluoro, —CN, —CF₃, —OCF₃, —OR⁶ and —NR⁶R⁷,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,
aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R³ to R⁵ when placed in adjacent positions together may form a bridge —(CR⁸R⁹)ₛ—O—(CR¹⁰R¹¹)ₜ—O—,
s is 0, 1 or 2,
t is 1 or 2,
R⁸, R⁹, R¹⁰ and R¹¹ independently are hydrogen, C₁₋₆-alkyl or fluoro,
D is —(CH₂)ₚ—, 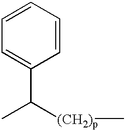
or —(CH₂)ₚ—O—,
p is 0, 1, 2, 3 or 4,
E is 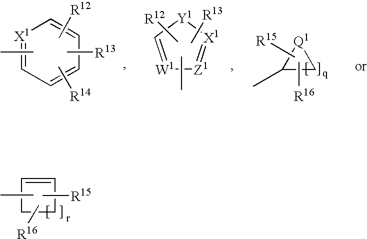
X¹, Z¹ and W¹ independently are —CH═ or —N═,
Y¹ is —O—, —S— or —NH—,
Q¹ is —CH₂— or —NH—,
q is 2, 3, 4, 5 or 6,
r is 1, 2, 3, 4 or 5,
R¹², R¹³ and R¹⁴ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷ R¹⁸, —NR¹⁷C(O)R¹⁸, —CH₂C(O)NR¹⁷R¹⁸, —OCH₂C(O)NR¹⁷R¹⁸, —OC(O)R¹⁷, —C(O)R¹⁷ or —C(O)OR¹⁷,
C₁₋₆-alkyl, C₂₋₆-alkenyl or C₂₋₆-alkynyl,
which may optionally be substituted with one or more substituents selected from fluoro, —CN, —CF₃, —OCF₃, —OR¹⁷ and —NR¹⁷R₁₈,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR¹⁷, —CN, —CF₃, —OCF₃, —OR¹⁷ and —NR⁷R¹⁸,
aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,
R¹⁷ and R¹⁸ independently are hydrogen or C₁₋₆-alkyl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹² to R¹⁴ when placed in adjacent positions together may form a bridge —(CR¹⁹R²⁰)ₓ—O—(CR²¹R²²)_{y}—O—,
x is 0, 1 or 2,
y is 1 or 2,
R¹⁹, R²⁰, R²¹ and R²² independently are hydrogen, C₁₋₆-alkyl or fluoro,
R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,
wherein the cyclic moieties may optionally be substituted with one or more substituents selected from halogen, —CN, —CF₃, —NO₂, —OR²³, —NR²³R²⁴ and C₁₋₆-alkyl,
R²³ and R²⁴ independently are hydrogen or C₁₋₆-alkyl, or
R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or E is
C₁₋₆-alkyl, C₂₋₆-alkenyl or C₂₋₆-alkynyl,
which may optionally be substituted with one or more substituents selected from halogen, —CN, —CF₃, —OCF₃, —NO₂, —OR²⁵,—SR²⁵, —NR²⁵R²⁶ and C₁₋₆-alkyl,
R²⁵ and R²⁶ independently are hydrogen or C₁₋₆-alkyl, or
R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
Z is —(CR²⁷R²⁸)ᵥ—(O)_{w}—(CR²⁹R³⁰)_{z}—,
v and z independently are 0, 1 or 2,
w is 0 or 1,
R²⁷, R²⁸, R²⁹ and R³⁰ independently are hydrogen or C₁₋₆-alkyl,
as well as any diastereomer or enantiomer or tautomeric form thereof including mixtures of these or a pharmaceutically acceptable salt thereof.

59. A method for delaying or preventing progression from non-insulin requiring type 2 diabetes to insulin requiring type 2 diabetes, said method comprising administering to a subject in need thereof an effective amount of a compound of formula (I′): 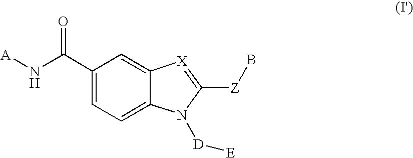
wherein
A is 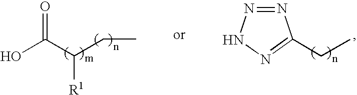
m is 0 or 1,
n is 0, 1, 2 or 3,
with the proviso that m and n must not both be 0,
R¹ is hydrogen, fluoro or —(CH₂)ₒ—OR²,
o is 0 or 1,
R² is hydrogen, C₁₋₆-alkyl, C₁₋₆-alkanoyl, aryl or aryl-C₁₋₆-alkyl,
X is —N═ or —CH═,
B is 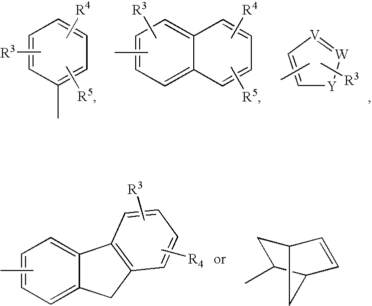
V and W independently are —CH═ or —N═,
Y is —O—, —S— or —NH—,
R³, R⁴ and R⁵ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR⁶, —NR⁶R⁷, —SR⁶, —NR⁶S(O)₂R⁷, —S(O)₂NR⁶R⁷, —S(O)NR⁶R⁷, —S(O)R⁶, —S(O)₂R⁶, —C(O)NR⁶R⁷, —OC(O)NR⁶R⁷, —NR⁶C(O)R⁷, —CH₂C(O)NR⁶R⁷, —OCH₂C(O)NR⁶R⁷, —OCH₂C(O)OR⁶, —OC(O)R⁶, —C(O)R⁶ or —C(O)OR⁶,
C₁₋₆-alkyl, C₂₋₆-alkenyl or C₂₋₆-alkynyl,
which may optionally be substituted with one or more substituents selected from fluoro, —CN, —CF₃, —OCF₃, —OR⁶ and —NR⁶R⁷,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR⁶, —CN, —CF₃, —OCF₃, —OR⁷, —NR⁶R⁷ and C₁₋₆-alkyl,
aryl, arylthio, aryl-C₁₋₆-alkylthio, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-C₁₋₆-alkyl, heteroaryl-C₂₋₆-alkenyl or heteroaryl-C₂₋₆-alkynyl,
of which the cyclic moieties may optionally be substituted with one or more substituents selected from halogen, —C(O)OR⁶, —CN, —CF₃, —OCF₃, —NO₂, —OR⁷, —NR⁶R⁷ and C₁₋₆-alkyl,
R⁶ and R⁷ independently are hydrogen or C₁₋₆-alkyl,
or R⁶ and R⁷ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R³ to R⁵ when placed in adjacent positions together may form a bridge —(CR⁸R⁹)ₛ—O—(CR¹⁰R¹¹)ₜ—O—,
s is 0, 1 or 2,
t is 1 or 2,
R⁸, R⁹, R¹⁰ and R¹¹ independently are hydrogen, C₁₋₆-alkyl or fluoro,
D is —(CH₂)ₚ—, 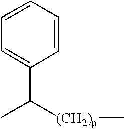
or —(CH₂)ₚ—O—,
p is 0, 1, 2, 3 or 4,
E is 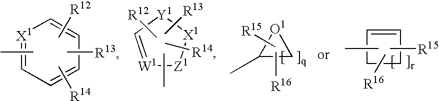
X¹, Z¹ and W¹ independently are —CH═ or —N═,
Y¹ is —O—, —S— or —NH—,
Q¹ is —CH₂— or —NH—,
q is 2, 3, 4, 5 or 6,
r is 1, 2, 3, 4 or 5,
R¹², R¹³ and R¹⁴ independently are
hydrogen, halogen, —CN, —CHF₂, —CF₃, —OCF₃, —OCHF₂, —OCH₂CF₃, —OCF₂CHF₂, —S(O)₂CF₃, —SCF₃, —NO₂, —OR¹⁷, —NR¹⁷R¹⁸, —SR¹⁷, —NR¹⁷S(O)₂R¹⁸, —S(O)₂NR¹⁷R¹⁸, —S(O)NR¹⁷R¹⁸, —S(O)R¹⁷, —S(O)₂R¹⁷, —C(O)NR¹⁷R¹⁸, —OC(O)NR¹⁷R¹⁸, —NR¹⁷C(O)R¹⁸, —CH₂C(O)NR¹⁷R¹⁸, —OCH₂C(O)NR¹⁷R¹⁸, —OC(O)R¹⁷, —C(O)R¹⁷ or —C(O)OR¹⁷,
C₁₋₆-alkyl, C₂₋₆-alkenyl or C₂₋₆-alkynyl,
which may optionally be substituted with one or more substituents selected from fluoro, —CN, —CF₃, —OCF₃, —OR¹⁷ and —NR¹⁷R¹⁸,
C₃₋₈-cycloalkyl, C₄₋₈-cycloalkenyl, heterocyclyl, C₃₋₈-cycloalkyl-C₁₋₆-alkyl, C₃₋₈-cycloalkyl-C₁₋₆-alkoxy, C₃₋₈-cycloalkyloxy, C₃₋₈-cycloalkyl-C₁₋₆-alkylthio, C₃₋₈-cycloalkylthio, C₃₋₈-cycloalkyl-C₂₋₆-alkenyl, C₃₋₈-cycloalkyl-C₂₋₆-alkynyl, C₄₋₈-cycloalkenyl-C₁₋₆-alkyl, C₄₋₈-cycloalkenyl-C₂₋₆-alkenyl, C₄₋₈-cycloalkenyl-C₂₋₆-alkynyl, heterocyclyl-C₁₋₆-alkyl, heterocyclyl-C₂₋₆-alkenyl, heterocyclyl-C₂₋₆-alkynyl,
of which the cyclic moieties may optionally be substituted with one or more substituents selected from fluoro, —C(O)OR¹⁷, —CN, —CF₃, —OCF₃, —OR¹⁷ and —NR¹⁷R¹⁸,
aryl, aryloxy, aryloxycarbonyl, aroyl, aryl-C₁₋₆-alkoxy, aryl-C₁₋₆-alkyl, aryl-C₂₋₆-alkenyl, aryl-C₂₋₆-alkynyl, heteroaryl, heteroaryl-Cl 6-alkyl, heteroaryl-C₂₋₆-alkenyl or heteroaryl-C₂₋₆-alkynyl,
of which the cyclic moieties may optionally be substituted with one or more substituents selected from halogen, —C(O)OR¹⁷, —CN, —CF₃, —OCF₃, —NO₂, —OR¹⁷, —NR¹⁷R¹⁸ and C₁₋₆-alkyl,
R¹⁷ and R¹⁸ independently are hydrogen or C₁₋₆-alkyl,
or R¹⁷ and R¹⁸ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or two of the groups R¹² to R¹⁴ when placed in adjacent positions together may form a bridge —(CR¹⁹R²⁰)ₓ—O—(CR²¹R²²)_{y}—O—,
x is 0, 1 or 2,
y is 1 or 2,
R¹⁹, R²⁰, R²¹ and R²² independently are hydrogen, C₁₋₆-alkyl or fluoro,
R¹⁵ and R¹⁶ independently are hydrogen, halogen, —CN, —CF₃, —OR²³, —NR²³R²⁴, C₁₋₆-alkyl, C₃₋₈-cycloalkyl, C₄₋₈-cycloalkenyl, aryl or aryl-C₁₋₆-alkyl,
wherein the cyclic moieties may optionally be substituted with one or more substituents selected from halogen, —CN, —CF₃, —NO₂, —OR²³, —NR²³R²⁴ and C₁₋₆-alkyl,
R²³ and R²⁴ independently are hydrogen or C₁₋₆-alkyl, or
R²³ and R²⁴ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
or E is
C₁₋₆-alkyl, C₂₋₆-alkenyl or C₂₋₆-alkynyl,
which may optionally be substituted with one or more substituents selected from halogen, —CN, —CF₃, —OCF₃, —NO₂, —OR²⁵,—SR²⁵, —NR²⁵R²⁶ and C₁₋₆-alkyl,
R²⁵ and R²⁶ independently are hydrogen or C₁₋₆-alkyl, or
R²⁵ and R²⁶ when attached to the same nitrogen atom together with the said nitrogen atom may form a 3 to 8 membered heterocyclic ring optionally containing one or two further heteroatoms selected from nitrogen, oxygen and sulfur, and optionally containing one or two double bonds,
Z is —(CR²⁷R²⁸)ᵥ—(O)_{w}—(CR²⁹R³⁰)_{z}—,
v and z independently are 0, 1 or 2,
w is 0 or 1,
R²⁷, R²⁸, R²⁹ and R³⁰ independently are hydrogen or C₁₋₆-alkyl,
as well as any diastereomer or enantiomer or tautomeric form thereof including mixtures of these or a pharmaceutically acceptable salt thereof.

60. The method according to claim 49, wherein the disease or disorder to be treated is hyperglycemia.

61. The method according to claim 49, wherein the disease or disorder to be treated is impaired glucose tolerance (IGT).

62. The method according to claim 49, wherein the disease or disorder to be treated is type 2 diabetes.

63. The method according to claim 49, wherein the disease or disorder to be treated is type 1 diabetes.

64. The method according to claim 49, wherein the disease or disorder to be treated is obesity.

65. The method according to claim 49, wherein the disease or disorder to be treated is dyslipidemia.

66. The method according to claim 49, said method further comprising treatment with an antidiabetic agent.

67. The method according to claim 49, said method further comprising treatment with an antiobesity agent.

68. The method according to claim 49, said method further comprising treatment with an antihyperlipidemic agent.

69. The method according to claim 49, said method further comprising treatment with an an- tihypertensive agent.
